(19) United States
(12) Patent Application Publication
(10) Pub. No.: US 2014/0088652 A1
(43) Pub. Date: Mar. 27, 2014

(54) INTERNAL DYNAMIC SPLINT AND METHOD FOR USE THEREOF

(71) Applicants: Justin M. Brown, San Diego, CA (US); Diana A. GLASER, San Diego, CA (US)

(72) Inventors: Justin M. Brown, San Diego, CA (US); Diana A. GLASER, San Diego, CA (US)

(21) Appl. No.: 14/035,648

(22) Filed: Sep. 24, 2013

Related U.S. Application Data
(60) Provisional application No. 61/706,580, filed on Sep. 27, 2012.

(51) Int. Cl. A61B 17/84

(52) U.S. Cl. CPC A61B 17/844
USPC 606/286; 606/326

(57) ABSTRACT

An implantable internal dynamic splint device that replaces or augments the function of a muscle that is antagonized at a joint by a functional muscle is described. The device comprises an elastic body, a first tissue-attaching component adapted to be attached to a bone, and a second tissue-attaching component adapted to be attached to a bone or a soft tissue. Also disclosed is a method for treating muscle function deficiency using the internal dynamic splinting device. The method comprises the steps of making an incision at a target site on a subject, attaching the first tissue-attaching component of the internal dynamic splinting device to a first bone, attaching the second tissue-attaching component of the internal dynamic splinting device to a second bone or a soft tissue, and closing the incision. A method for compensating muscle function deficiency on a side of a joint using the device is also disclosed.

This application claims priority to U.S. Provisional Application No. 61/706,580, filed Sep. 27, 2012, the entirety of which is incorporated herein by reference.

### FIELD

This application generally relates to medical devices. In particular, the application relates to implantable dynamic splints for moving a portion of a limb.

### BACKGROUND

Injuries occurred with falls, motor vehicle accidents, or workplace accidents, can lead to partial paralysis of a limb. For example, the peroneal nerve in the lower leg, which is responsible for active dorsiflexion of the ankle, is frequently injured due to its vulnerable superficial location just below the knee. Additionally, spine degeneration over time or acutely with spinal trauma, can lead to situations as severe as quadriplegia with only partial use of the upper limbs remaining or as mild as foot drop or hand weakness.

In foot drop, the patient may suffer from weakness and imbalance of the muscles around the ankle joint, having profound effect on gait. Treatment options are meager, most commonly making the patient dependent on a static external brace to pull the foot up and create a more functional gait pattern. Other solutions involving complicated neurostimulation platforms have not been widely accepted and have no role in patients whose primary injury is of the nerve or muscle.

Similarly, in radial nerve palsy, when the wrist and fingers cannot be extended, an external dynamic splint will hold the wrist in a neutral position while the fingers are suspended in an extended position by rubber bands. The patient can easily overcome these rubber bands with normal finger flexion, yet the fingers spring back once the patient relaxes as if the finger extensors were present and functional.

Dynamic splinting solutions have been developed for numerous injuries, such as those resulting in wrist drop, foot drop, triceps paralysis, and pronation deficits among others. These devices are used to guide therapy and preserve range of motion when some degree of recovery is expected. Non-operative options include external splinting devices, such as braces, which are cumbersome, difficult to place without assistance and often uncomfortable. Alternatively, operative options are limited to tendon transfer, which is a surgical procedure in which a muscle tendon from one functional muscle group is used to replace a tendon of the paralyzed muscle group, achieving some movement. This treatment allows for some function of the paralyzed muscle group, but it also decreases the strength of the non-paralyzed muscle group. Variations of the tendon transfer procedure are mainly limited to variation of which tendon is to be transferred and the method of reattachment. There are many cases in which a good tendon transfer option is not available and long term bracing is required.

### SUMMARY

One aspect of the present application relates to an internal dynamic splinting device. The device comprises an elastic body; a first tissue-attaching component connected to the elastic body, wherein the first tissue-attaching component is adapted to be attached to a bone; and a second tissue-attaching component connected to the elastic body, wherein the second tissue-attaching component is adapted to be attached to a bone or a soft tissue.

Another aspect of the present application relate to a method for treating a condition involving muscle function deficiency or for compensating muscle function deficiency on a side of a joint. The method comprises the steps of making an incision at a target site on a subject requiring such treatment; attaching the first tissue-attaching component of the internal dynamic splinting device of the present application to a first bone; attaching the second tissue-attaching component of the internal dynamic splinting device of claim 1 to a second bone or a soft tissue; and closing the incision.

In several embodiments, the splinting device is characterized by first tissue-attaching component of the internal dynamic splinting device of the present application to a first bone on the deficiency side of the joint, and attaching the second tissue-attaching component of the internal dynamic splinting device of the present application to a second bone or a soft tissue on the deficiency side of the joint, separated by the internal dynamic splinting device which is stretched to the extended length when an antagonist muscle on an opposite side of the joint contracts, and wherein the internal dynamic splinting device returns to its resting length when the antagonist muscle on the opposite side relaxes.

### BRIEF DESCRIPTION OF THE DRAWINGS

FIGS. 1A-C show one exemplary embodiment of the present device having a spring inside a cylinder-shaped housing.

FIG. 2 shows an exploded view of the embodiment depicted in FIGS. 1A-C.

FIGS. 3A-B show a detailed view of elements of the embodiment depicted in FIGS. 1A-C.

FIGS. 4A-B show perspective views of external and internal components of the embodiment depicted in FIGS. 1A-C.

FIGS. 5A-D show detailed views of a fixation plate component of the embodiment depicted in FIGS. 1A-C.

FIGS. 6A-D show detailed views of a spring housing component of the embodiment depicted in FIGS. 1A-C.

FIGS. 7A-D show detailed views of a cap component of the embodiment depicted in FIGS. 1A-C.

FIGS. 8A-D show detailed views of a rod component of the embodiment depicted in FIGS. 1A-C.

FIG. 9 shows a second exemplary embodiment of the present device having a spring inside a cylinder-shaped housing.

FIGS. 10A-B show perspective views of external and internal components of the embodiment depicted in FIG. 9.

FIGS. 11A-D show detailed views of a fixation plate component of the embodiment depicted in FIG. 9.

FIGS. 12A-D show detailed views of a spring housing component of the embodiment depicted in FIG. 9.

FIGS. 13A-D show detailed views of a buckle component of the embodiment depicted in FIG. 9.

FIGS. 14A-D show detailed views of an anchor component of the embodiment depicted in FIG. 9.

FIGS. 15A-C show detailed views of a gasket component of the embodiment depicted in FIG. 9.

FIGS. 16A-D show detailed views of a cap component of the embodiment depicted in FIG. 9.

FIGS. 17A-B show perspective views of external and internal components a multi-chambered embodiment of the embodiment depicted in FIG. 9.

FIGS. 18A-D show detailed views of the multi-chambered embodiment depicted in FIGS. 17A-B.

FIGS. 19A-C show exemplary embodiments of attachment between a tether and the distal end of a rod that attaches to a spring.

FIG. 20 shows one embodiment of an exemplary elastic embodiment of the present device, attaching an elastic element that is fixed to a long bone of a forelimb to a tendon attached to an extremity.

FIG. 21 shows another embodiment of an exemplary elastic embodiment of the present device, attaching an elastic element that is fixed to a long bone of a forelimb to a fixed point implanted into a bone of an extremity.

FIG. 22 shows another exemplary embodiment of the present device, having attachment to the tendons on the dorsal surface of the fingers.

FIG. 23 shows another view of the embodiment depicted in FIG. 8.

FIGS. 24A-D show detailed views of elements of the embodiment depicted in FIG. 8.

FIGS. 25A-D show an exemplary attachment of tendons to the embodiment depicted in FIG. 8.

FIGS. 26A-C show an embodiment of an elastic tether, such as the one depicted in FIGS. 20 and 21.

FIGS. 27A-C show another embodiment of an elastic tether, such as the one depicted in FIGS. 20 and 21.

FIG. 28 shows an exemplary attachment of the elastic tether.

FIG. 29 shows another exemplary attachment of the elastic tether.

FIG. 30 shows another embodiment of an elastic body.

FIG. 31A-C show an embodiment of attachment of the present device to a bone.

FIGS. 32A-D show embodiments of sealing mechanisms for the housing.

FIGS. 33A-D show another embodiment of the attachment between a tether and a rod that attaches to a spring.

### DETAILED DESCRIPTION

The following detailed description is presented to enable any person skilled in the art to make and use the device of the present application. For purposes of explanation, specific nomenclature is set forth to provide a thorough understanding of the present application. However, it will be apparent to one skilled in the art that these specific details are not required to practice the subject of the application. Descriptions of specific applications are provided only as representative examples. The present application is not intended to be limited to the embodiments shown, but is to be accorded the widest possible scope consistent with the principles and features disclosed herein.

This description is intended to be read in connection with the accompanying drawings, which are to be considered part of the entire written description of this application. The drawing figures are not necessarily to scale and certain features of the application may be shown exaggerated in scale or in somewhat schematic form in the interest of clarity and conciseness. In the description, relative terms such as “front,” “back,” “up,” “down,” “top,” “bottom,” “upper,” and “lower,” as well as derivatives thereof, should be construed to refer to the orientation as then described or as shown in the drawing figure under discussion. These relative terms are for convenience of description and normally are not intended to require a particular orientation. Terms concerning attachments, coupling and the like, such as “engaged,” “connected,” “mounted,” and “attached,” refer to a relationship wherein structures are secured or attached to one another either directly or indirectly through intervening structures, as well as both movable or rigid attachments or relationships, unless expressly described otherwise. In some embodiments, a first component that is “connected” to a second component can be an integral part, or a section, of the second component.

As used herein, the term “proximal” or derivatives thereof refers to the direction on an arm or leg, or of a device placed on the arm or leg, toward the trunk of the body. Conversely, as used herein, the term “distal” or derivatives thereof refers to the direction on an arm or leg, or of a device placed on the arm or leg, away from the trunk of the body, or towards the fingers or toes, respectively.

As used herein, the term “appendage” refers to an arm, leg, hand or foot. As used herein, the term “limb” refers to an arm, leg, forearm or lower leg. As used herein, the terms “digit” and “phalanges” refers to fingers, thumbs and toes.

As used herein, the term “flexor” and derivatives thereof refers to a muscle or muscle group that when contracted acts to bend a joint or limb in the body, reducing the angle between the bones on the opposite sides of the joint, for example the closing of the hand or the lifting of the foot in a proximal direction. As used herein, the term “extensor” and derivatives thereof refers to a muscle or muscle group that extends or straightens a limb or body part, increasing the angle between the bones on the opposite sides of the joint, for example the opening of the hand or the extending of the foot in a distal direction. As used herein, the term “rotator” and derivatives thereof refers to a muscle or muscle group serving to rotate a part of the body.

As used herein, the term “elastic” refers to an element of a device that is capable of returning towards its original shape after compression, expansion, stretching, or other

### Internal Dynamic Splinting Device

One aspect of the present application relates to an internal dynamic splinting device. The device comprises an elastic body having a first end and a second end that defines a resting length between the first end and the second end, a first tissue attaching component connected to the elastic body and a second tissue-attaching component connected to the elastic body. The elastic body has a resilience to extend to a stretched length when a stretching force is applied to the first end and/or the second end of the elastic body, and to return from the stretched length to the resting length after the removal of the stretching force. The first tissue-attaching component is adapted to be attached to a bone and the second tissue-attaching component is adapted to be attached to a bone or a soft tissue.

In some embodiments, the internal splinting device of this application is a stretchable/elastic device which is extended by active muscle force and returns to its original state after the extension force is being removed.

In some embodiments, the internal splinting device of this application is implanted on the tendon of a non-functional, paralyzed muscle or muscle group responsible for movement of the joint in one direction such as the tendon to flex the ankle (foot drop) or the one that extends the wrist (wrist drop) or implanted directly on the bony part of the foot or wrist. The device generates an action similar to a spring, pulling the limb in a passive motion. The opposite motion of the device (ankle extension or wrist/finger flexion) is performed by the non-paralyzed muscle group.

In some embodiments, the stretched length is at least 5%, 10%, 15%, 20%, 25%, 30%, 35%, 40%, 50%, 60%, 70% or 80% greater than the resting length. In other embodiments, the stretched length is about 5-10%, about 5-15%, about 5-20%, about 5-30%, about 5-40%, about 5-50%, about 5-60%, about 5-70%, about 5-80%, about 5-90% or about 5-100% greater than the resting length.

### Elastic Body

The elastic body is a stretchable body comprising a material with elastic properties, such as an elastic band or silicon, or an element that generates spring action, such as a spring, or a hydraulic or gas mechanism that will move cylinders in and out of each other, or any other mechanism or material that generates an elastic or stretchable action of the elastic body and its attachment ends.

In some embodiments, the elastic body is composed of a non-encased elastic in a stand-alone configuration. The elastic body may comprise one or more elastic bands. In some embodiments, the elastic body contains a single elastic band with a desired stiffness or elasticity. In other embodiments, the elastic body contains two or more elastic bands that form a bundle. The multiple bands in the bundle may have the same or different stiffness. The advantage of having a bundle of elastic bands is that one can adjust the stiffness during surgery by reducing or increasing the number of elastic bands in the bundle. In some embodiments, the elastic band further comprise a braid of multiple strings. The elasticity of the elastic band is determined by the elasticity of the individual strings and the pattern the multiple strings are braided. A preferred material for use as a braid is a polymer, such as polyester, polyethylene, or Kevlar. Other materials that may be used include metals such as stainless steel, or suitable metal alloys.

In other embodiments, the elastic body comprises a housing and an elastic component within the housing. The housing can be of various shape and size. In some embodiments, the housing is a tubular housing having a cross sectional profile of a circle, a circle with a flattened side, an oval or an oval with a flattened side. The housing may be fabricated from any suitable material. In some embodiments, the housing is made of a suitable metal. Examples of suitable metals include, but are not limited to, stainless steel, titanium and titanium alloys, tantalum and tantalum alloys, cobalt-chromium alloys, zirconium and zirconium alloys, nickel and nickel alloys. In other embodiments, the housing is made from, or comprises, a biocompatible non-degradable polymeric material. Examples of biocompatible non-degradable polymeric materials include, but are not limited to, hydrogels, polyurethanes, polyvinyl alcohol (PVA), polyester (e.g., Dacron®), polyethylene, polyaramid, poly-paraphenylene terephthalamide (e.g., Kevlar®), polyethylene oxide (PEO), PEO based polyurethane polyvinylpyrrolidone (PVP), polyacrylamide, polyethylene terephthalate, acrylic polymers, methacrylic polymers, polyurea, polyolefin, halogenated polyolefin, polysaccharide, vinylic polymer, polyphosphazene, polysiloxane, polypropylene (PP), poly(tetrafluroethylene) (PTFE), poly(methymethacrylate), polyetheretherketone (PEEK) and the like. In some embodiments, the housing comprises elastomers such as silicone, polyurethane, or polyester (e.g., Hytrel®), reinforced with a fiber, such as polyethylene (e.g., ultra high molecular weight polyethylene, UHMWPE), polyethylene terephthalate, or poly-paraphenylene terephthalamide (e.g., Kevlar®).

In some embodiments, the housing comprises a layer of flexible material to enhance the bending, flexion, and extension of the housing. The layer may comprise any one or more of the materials described above, including hydrogels, polyurethanes, polyvinyl alcohol (PVA), polyethylene oxide (PEO), polyvinylpyrrolidone (PVP), polyacrylamide, PEO based polyurethane, elastomers such as silicone, polyurethane, or polyester (e.g., Hytrel®), reinforced with a fiber, such as polyethylene (e.g., ultra high molecular weight polyethylene, UHMWPE), polyethylene terephthalate, or poly-paraphenylene terephthalamide (e.g., Kevlar®). In some embodiments, the layer of flexible material is made of polyurethane or silicone. The layer of flexible material may be fabricated by injection molding, two-part component mixing, or dipping the housing into a polymer solution. A function of the layer of flexible material is to act as a barrier that keeps natural in-growth outside the device. In some embodiments, the layer of flexible material is stretchable. In some embodiments, the housing is stretchable. In some embodiments, the elastic body of the device of the present application comprises stretchable housing and a spring located inside the housing. The housing is stretchable to the same extent as the spring.

In some embodiments, the housing is made from or comprises a fiber material, such as carbon or glass fibers. In some embodiments, the fibers are processed (e.g., injection molded or extruded) with an elastomer to encapsulate the fibers, thereby providing protection from tissue ingrowth and improving torsional and flexural stiffness. In other embodiments, the fibers are coated with one or more other materials to improve fiber stiffness and wear. In other embodiments, the fibers may be terminated on the first and second tissue-attaching components by tying a knot in the fiber on the surface of a tissue-attaching component. Alternatively, the fibers may be terminated on a tissue-attaching component by slipping the terminal end of the fiber into a slot on an edge of the tissue-attaching component, similar to the manner in which thread is retained on a thread spool. The slot may hold the fiber with a crimp of the slot structure itself, or by an additional retainer such as a ferrule crimp. As a further alternative, tab-like crimps may be machined into or welded onto the structure of the tissue-attaching component to secure the terminal end of the fiber. The fiber may then be closed within the crimp to secure it. As a still further alternative, a polymer may be used to secure the fiber to the tissue-attaching component by welding. The polymer would preferably be of the same material as the fiber (e.g., PE, PET, or the other materials listed above). Still further, the fiber may be retained on the tissue-attaching component by crimping a cross-member to the fiber creating a T-joint, or by crimping a ball to the fiber to create a ball joint.

In other embodiments, the housing is made from a biocompatible non-degradable silicon material. In some embodiments, the outer surface of the housing is coated, for example with a material that inhibits or prevents the attachment or ingrowth of bodily tissue onto/into the housing. In some embodiments, the housing is composed of a woven material. In a further embodiment, the woven material is treated to prevent ingrowth of bodily tissue. In yet other embodiments, the housing is made from a ceramic material or a composite comprising ceramic material. In some embodiments, the housing is sealed to the outer environment to prevent ingrowth. Various sealing mechanisms, such as o-rings, buffer seals, rod seals, piston seals, other seals and combinations thereof, may be employed. The seals may be operated at regular pressure, high pressure or ultra-high pressure.

In some embodiments, the elastic component comprises a spring. In some embodiments, the spring changes from a resting position to a stretched position when the elastic body extends from said resting length to said stretched length. In some other embodiments, the spring is a clock spring or power spring, that rolls up automatically to its original position when force is eliminated. In some other embodiments, the spring is a compression spring that is compressed from its resting position when the elastic body extends from the resting to the stretched lengths. In some other embodiments the compression spring can be a wave spring or other spring shape.

In some embodiments, the elastic body further comprise a housing cap that engages with the first tissue-attaching component. In one embodiment, the housing cap is located on one end of the housing and is engaged with the spring inside the housing. In some embodiments, the elastic body comprises a housing and an elastic component. The elastic component further comprises a spring and a rod attached to the spring. The rod is connected to the second tissue-attaching component through a tether. The tether is a non-elastic robe, wire or cable that is attached to the rod on one end and to a bone/ or soft tissue on the other end through the second tissue-attaching component, which can be a section of the tether. Such a design prevents friction and allows for better sealing of the housing. In one embodiment, the tether is attached to the short screw head part. The screw head part is screwed on the rod which is attached to the spring. This design allows for easy exchange of the tether in case the tether wears down after certain period of use. In other embodiments, the spring is connected on one end to a screw mechanism that allows adjustment of the spring stiffness. In another embodiments, the rod comprises a carabiner type end and the tether has a loop at the end allowing easy connection or disconnection from the rod. These variations allow adjustment of the stiffness of the elastic body during implantation and thus the individualization of the device.

In other embodiments, the spring changes from a resting position to a compressed position when the elastic body extends from the resting length to the stretched length. In some embodiments, the elastic body further comprises a sealing cap at one end of said housing and a tether having a knob on one end. The knob is located inside the housing and the spring is located between the knob and the sealing cap. In some embodiments, the elastic body further comprises a device for adjusting the resting length of the elastic body and tension on the tether.

In some embodiments, the elastic body comprises two or more tubular or cylinder-shaped housings. Each housing contains an elastic component, such as a spring. In other embodiments, the elastic body comprises a single housing with two or more tubular or cylinder-shaped chambers. Each chamber contains an elastic component, such as a spring. The multiple elastic components in these embodiments may be attached to different tendons or tissues at their distal end so as to provide a balanced retraction force. For example, a splint device with two or more springs would allow connection to two or more points in the foot of a foot drop patient and prevent a tilt of the foot.

### Tissue-Attaching Components

The first and second tissue-attaching components are designed to attach or fix the device of the present application to a bone, such as a long bone on a limb, or a soft tissue, such as a tendon. In some embodiments, the first tissue-attaching component comprises a plate having at least one hole for mounting a fastening device that fastens the plate to a bone and a clip or bracket that retains the housing. Such fastening devices are well known in the art. In some embodiments, the first tissue-attaching component is fastened or fixed to a long bone of an extremity. Fixation to the long bone of the extremity can involve one or multiple points of fixation with, for example, screws or by any other means known in the art for fixation of a plate or brace to a long bone. For example, fixation can be achieved by methods similar to the fixation of a distal radius fracture. The number of screws and the details of the fixation can vary depending on the load bearing demand upon the system. In some embodiments, fixation is with 2-8 screws. In some embodiments, the screws are positioned in angled directions to allow for better grip and fixation between the plate and the bone. In some embodiments, the fixation stabilizes a base plate to which the rest of the device is affixed.

In some embodiments, the first tissue-attaching component comprises a plate that will be attached to the bone. The plate has rails to allow the elastic body to be moved up and down the plate during implantation. The elastic body is designed to have a catching structure that fits in the rails of the plate and allows movement along the rails. Once an optimal position of the elastic body is identified (i.e., a position of the elastic body that provides desired stiffness of the device based on patient specific characteristics), the elastic body is screwed or otherwise secured into the rail to fix the position.

In some embodiments, the second tissue-attaching component is adapted to attach to a soft tissue, such as a tendon. In some embodiments, the second tissue-attaching component comprises a rod having a distal end and proximal end. The rod is engaged with the spring at the proximal end and has a hole at the distal end to facilitate attachment to the soft tissue.

In other embodiments, the first tissue attachment component engages with a bone anchor that is fastened to a bone. The second tissue-attachment component comprises a rod having a distal end and proximal end. The rod is engaged with the elastic component at the proximal end and has a hole at the distal end to facilitate attachment to a soft tissue.

The first tissue attachment component and the second tissue attachment component can be made from any suitable materials, such as metal, ceramic, biocompatible polymers, and woven fibers.

In cases where the elastic body is composed of a non-encased elastic band or a bundle of elastic bands in a stand-alone configuration, the first tissue attachment component and the second tissue attachment component can be part of the elastic body itself. Alternatively, the elastic band or bundle of bands may be attached on either or both ends to a fixation plate or a bone anchor and then fastened to a bone. In one embodiment, the elastic band passes through a bone tunnel in the bone with the fixation plate attached to the bone on one end of the bone tunnel and the elastic device attached to the bone or soft tissue on the other end of the bone tunnel. In another embodiment, the elastic band or bundle wraps around the bone and attaches on one end with a plate to the back of the bone and on the other end to the soft tissue or bone.

Similar to the housing, other components of the internal splinting device of the present application can be made from suitable materials described above, such as metal, ceramics, silicone and a biocompatible non-degradable polymeric materials.

In some embodiments, the internal splinting device of this application is motorized. In some embodiments, the device is actively electrically controlled. In other embodiments, the device is passively electrically controlled. In some embodiment, the device is programmable. In some other embodiments, the internal splinting device of this application further comprises an actuator that operates under hydraulic or pneumatic pressure and controls movement.

Another aspect of the present application relates to an implantable splinting device. The device comprises (1) an elastic body having a proximal end and a distal end, the elastic body comprises a housing, a spring resided inside the housing, and a rod connected to the spring, wherein the rod extends outside the housing from the distal end of the housing, (2) a first tissue-attaching component connected to the proximal end of the elastic body, wherein the first tissue-attaching component is adapted to be attached to a bone, (3) a tether connected to the rod; and (4) a second tissue-attaching component connected to the tether, wherein the second tissue-attaching component is adapted to be attached to a bone or a soft tissue.

In some embodiments, the implantable splinting device further comprises a tension adjustment component that controls tension of the spring. In some related embodiments, the tension adjustment component is located inside the housing. In other related embodiments, the tension adjustment component is located outside the housing.

In some embodiments, the tether is a non-elastic tether. In other embodiments, the tether is an elastic tether.

### Methods of Using the Internal Dynamic Splinting Device

Another aspect of the present application relates to a method for treating a condition involving muscle function deficiency using the internal dynamic splinting device of the present application. The method comprises making an incision at a target site on a subject requiring such treatment, attaching the first tissue-attaching component of the internal dynamic splinting device to a first bone, attaching the second tissue-attaching component of the internal dynamic splinting device to a second bone or a soft tissue and closing the incision. In some embodiments, the internal dynamic splint device is implanted by full open surgery, opening the skin of the subject over the entire length of the device. In other embodiments, the device is implanted by an arthroscopic or endoscopic surgery. In some embodiments, the device is placed with a minimally invasive procedure. For example, the subject's skin is opened only in the regions of the proximal attachment point and the distal attachment point, with the elastic element or tether of the device being routed under the skin of the subject between the attachment points, such as through a cannula.

In some embodiments, the condition involving muscle function deficiency is a condition of paralysis in which movement of one muscle group is unopposed. Examples of such conditions include conditions of paralysis resulted from muscle injuries, peripheral nerve injuries, congenital conditions, lower spinal injuries, cervical spinal cord injuries or stroke and brain injuries.

Another aspect of the method of the present application relates to a method for compensating muscle function deficiency on a side of a joint. The method comprises the steps of attaching the first tissue-attaching component of the internal dynamic splinting device of the present application to a first bone on the deficiency side of the joint; and attaching the second tissue-attaching component of the internal dynamic splinting device of the present application to a second bone or a soft tissue on the deficiency side of the joint, wherein the internal dynamic splinting device is stretched to the stretched length when an antagonist muscle on an opposite side of the joint contracts, and wherein the internal dynamic splinting device returns to its resting length when the antagonist muscle on the opposite side relaxes.

The method of the present application can also be used as treatment options for patients with nerve or muscle damage affecting the muscle group on one side of a joint, while the muscle group on the other side of the joint is still functional. The internal dynamic splint of the present invention employs an implantable system that can be fixated to a bone of the extremity that is proximal to the joint to be operated by the device. The device has an elastic or spring-loaded mechanism that can be fixated to the tendons of interest to allow for displacement within the normal range of motion of the joint and that recoils to its resting position once the antagonist muscle group relaxes.

Examples of conditions treatable with the present device include radial nerve palsy or damage to the nerves or muscles of the posterior radio-ulnar region, which can result in wrist drop and/or the inability to extend the fingers following the relaxation of their antagonists, the anterior radio-ulnar muscles.

The device of the present application has utility in conditions of paralysis in which movement of one muscle group is unopposed. Such conditions include, but are not limited to: muscle injury, such as compartment syndrome, necrosis of the muscle of a limb, traumatic soft tissue injury, a wound from a projectile or penetrating object (such as a bullet, arrow, dart, knife, razor, glass or any item that can inflict a similar wound); a peripheral nerve injury, which is a common cause of foot drop, but can also result in inability, as non-limiting examples, to push the foot down, extend the wrist, extend the fingers, flex at the elbow, extend at the elbow, externally rotate the shoulder or abduct the shoulder; congenital conditions including, but not limited to spina bifida (which typically causes compromised ambulation from dysfunction of the lower nerve roots of the spine), loss or impairment of hip extension, knee flexion or foot plantar flexion.

The device of the present application is also useful for the treatment of lower spinal injuries, including, but not limited to, cauda equina syndrome. Cervical spinal cord injuries typically leave a patient with some shoulder function and elbow flexion and possibly wrist extension, typically without elbow extension, shoulder adduction and hand function, all of which are treatable with the present internal dynamic splint device. In some cases, reconstructive hand surgery that restores function to a limited extent with tendon transfers and joint fusion can be further augmented with the present device to provide “active” finger extension recoil, which is frequently not possible with current reconstructive procedures.

The device of the present application may also be used to treat conditions resulting from stroke or brain injuries. Stroke and brain injuries often leave a hand without the ability to extend the wrist or fingers, while latent ability to flex both the wrist and fingers actively exists, but is masked. The present device can pull the hand into a functional position, allowing the masked ability to flex the wrist and fingers to become a useful function, dramatically improving their quality of life. Stroke patients frequently also suffer from an “equinovarus foot” that, like the hand, has a severe imbalance of function. The present device restores/augments foot eversion and dorsiflexion and allows the patient to place their foot flat on the floor, eliminating the need for an inflexible ankle foot orthosis, as is most commonly used now.

The implantable internal dynamic splint device of the present application can also be used to treat muscle function deficiencies in conditions including, but not limited to, polio, multiple sclerosis, cerebral palsy, paralysis related to West Nile virus, patients who have had successful brain tumor resections who have long life expectancy but with notable functional impairments, military personnel or civilians suffering the effects of blast injuries which have destroyed a region of muscle and nerve, patients with partial recovery from Guillian Bane syndrome or severe peripheral neuropathy.

The present application provides an alternative treatment which will overcome the disadvantages associated with current treatments and provide a simple but effective solution for the best joint movement. The implantable internal dynamic device described herein is useful for the treatment of patients with symptoms related to muscle or peripheral nerve trauma, as well as partial spinal cord injury. Such conditions include, but are not limited to, foot drop, wrist drop, radial nerve palsy, inability to extend one or more fingers and/or the thumb, triceps paralysis, pronation deficits and quadriceps paralysis or other conditions that inhibit the ability to extend the lower leg.

For example, in regard to foot drop, the device of the present application will be of a help for those patients who have a nerve injury and have lost the ability to dorsiflex their ankle but are still able to perform plantar flexion normally. An embodiment of the device is implanted on the dorsiflexor tendon and consists of internal elastic mechanism allowing for displacement within the normal range of motion of the joint. The system recoils to its resting position once the antagonist group relaxes, much like the dynamic splints. Therefore, ankle plantar flexion will be actively achieved by the patient's own non-paralyzed muscle groups, but the device will passively support the ankle in performing the dorsiflexion, which involves the paralyzed muscles leading to the foot drop condition.

Devices of the present application can be used to provide forearm pronation, foot dorsiflexion, triceps function/elbow extension, knee extension, etc. Essentially in any situation in which one muscle group is functional and its antagonist is not, this may have a role in increasing the functionality of that extremity. Additional applications include, but are not limited to: brachial plexus injury, and lumbosacral plexus injury. Devices of the present application are also useful for the treatment of any individual nerve injury, including, but not limited to, median, radial, ulnar, musculocutaneous, tibial, peroneal, femoral, cervical nerve roots and lumbar nerve roots.

Devices of the present application are also useful for the treatment of spinal cord injury, providing, by way of non-limiting example, finger flexion, forearm pronation, wrist extension and triceps function. Furthermore, the present device is useful for the treatment of stroke, providing improved wrist dorsiflexion, finger extension and foot dorsiflexion, as well as multiple sclerosis, Guillan-Barre syndrome, myasthenia gravis or any other nervous system or autoimmune disease related paralysis. Devices of the present application may also be useful in the treatment of diminished joint function in arthritic conditions.

In some embodiments, a device of the present application takes the place of, or supplements the action of, an extensor muscle or muscle group and is antagonistic to the function of a flexor muscle or muscle group. In other embodiments, the device takes the place of, or supplements the action of, a flexor muscle or muscle group and is antagonistic to the function of a extensor muscle or muscle group. In still other embodiments, the device takes the place of, or supplements the action of, a rotator muscle or muscle group and is antagonistic to the function of another rotator muscle or muscle group.

In some embodiments, the device of the present application is used in facial palsy and cosmetic applications providing tone to the face, lift of the brow, eye sphincter function, and/or mouth sphincter function. In other embodiments, the device of the present application is used in genitourinary applications providing urinary or anal sphincter function. In yet other embodiments, the device of the present application is used in orthopedic indications in which joints are not stable as a result of muscle weakness or ligament laxity. In this circumstance the device could stabilize a joint, provide balance about a joint, reduce a chronic joint subluxation. The device could also be used in conditions of muscle weakness where the muscle group is not actually paralyzed but provide insufficient drive to overcome the antagonist or balance forces across a joint.

### Spring-Loaded Internal Dynamic Splint Devices

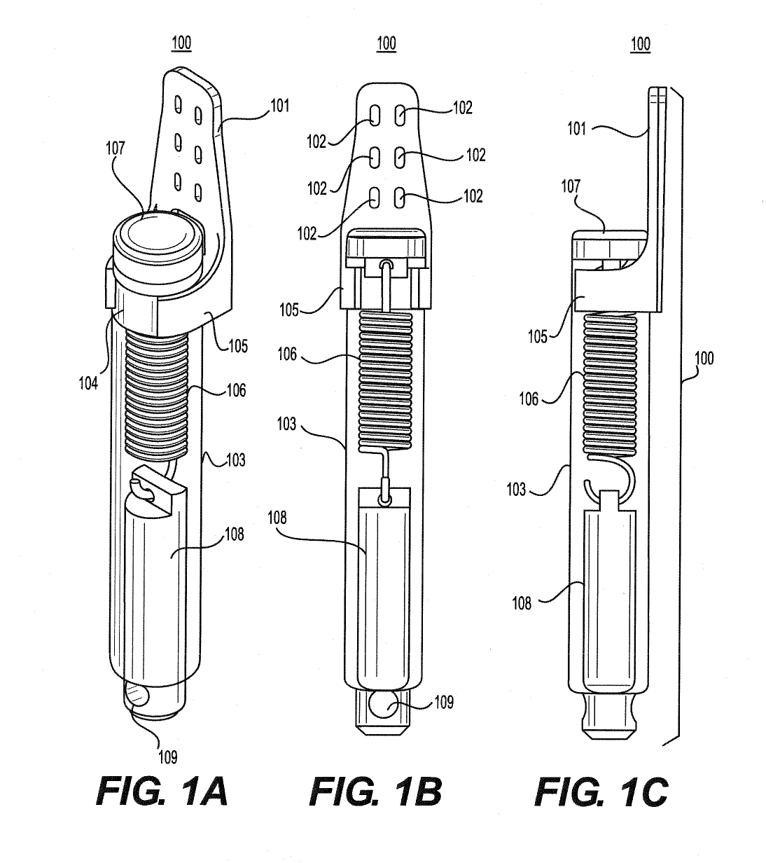

FIGS. 1A-C depict angled, front-facing and side views, respectively, of one embodiment of the device of the present application. This exemplary device 100 employs a spring 106 which is allowed to extend when the antagonist muscle group whose action is countered by the device contracts. The spring 106 then contracts in order to pull an appendage or digit back to a resting or neutral position upon relaxation of the antagonist muscle.

The device 100 comprises a fixation plate 101 that attaches to a bone, preferably a long bone, in the section of a limb that is distal to the joint(s) whose operation is to be effected by the action of the device 100. For example, in the case of foot drop, where operation of the ankle by the device 100 is desired in order to provide the antagonistic motion to the action of the posterior tibio-fibular muscles, the fixation plate 101 is attached to the anterior surface of the tibia. In the case of wrist drop or the inability to extend fingers, where operation of the wrist or fingers by the device is desired in order to provide the antagonistic motion to the action of the anterior radio-ulnar muscles, the fixation plate 101 is attached to the posterior surface of the radius. In some cases of where the patient is only unable to extend the thumb, the fixation plate 101 may be attached to the posterior surface of the ulna.

Still referring to FIGS. 1A-C, the fixation plate 101 is secured to a long bone by the insertion of a fastening device through at least one hole 102 in the fixation plate 101. In some embodiments the fixation plate 101 has 1, 2, 3, 4, 5, 6, 7, 8, 9, 10 or more holes 102. In a particular embodiment, the fixation plate 101 has 4-6 holes. The number of holes and fastening devices used is dependent upon the location and size of the device, as well as the load placed upon the device by the movement of the appendage or digit. In some embodiments, a fastening device is inserted through each of the holes 102 in the fixation plate 101. In other embodiments, at least one hole 102 does not have a fastening device inserted there through. In particular embodiments, the pattern of which holes have an inserted fastening device and which holes do not may be dependent upon whether the device 100 is employed on a left or right limb of the patient's body.

The device 100 further comprises a cylinder-shaped spring housing 103. In some embodiments, the spring housing 103 comprises at least one groove 104 that secures the spring housing 103 between brackets 105 on the fixation plate 101, although any method or means for securing the spring housing 103 to the fixation plate 101 of the device 100. In some embodiments, the spring housing 103 comprises means for directly attaching the spring housing 103 to a long bone without the use of a separate attachment component, for example flanges with holes for fastening device or straps that encircle and secure to the long bone. In some embodiments, the device 100 comprises a single spring housing 103.

In other embodiments, dependent upon the application, the device 100 comprises two spring housings 103, wherein each spring housing comprises a spring that is functionally attached to a separate tendon, or is functionally tethered to opposite sides of the foot to aid in foot leveling. In still other embodiments, dependent upon the application, the device 100 comprises 2, 3, 4 or 5 spring housings 103, for example, when each spring housing 103 comprises a spring that is functionally attached to a separate digit of the hand, for example to allow the separate extension of each connected digit. In other embodiment, the device 100 comprises a single spring housing 103 that contains two or more cylinder-shaped chambers. Each chamber harbors a spring that is functionally attached to a separate tendon, or is functionally tethered to opposite sides of the foot to aid in foot leveling. The chambers may be arranged in parallel or in series.

The fixation plate 101, spring housing 103, sealing cap 107 and rod 108 can be made from any medically acceptable material, such as metal, ceramic, biocompatible non-degradable polymeric materials, fibers and woven fibers. Examples of suitable metals include, but are not limited to, stainless steel, titanium and titanium alloys, tantalum and tantalum alloys, cobalt-chromium alloys, zirconium and zirconium alloys, nickel and nickel alloys. Examples of biocompatible non-degradable polymeric materials include, but are not limited to, polyester (e.g., Dacron®), polyethylene, polyaramid, poly-paraphenylene terephthalamide (e.g., Kevlar®), polyethylene terephthalate, acrylic polymers, methacrylic polymers, polyurethane, polyurea, polyolefin, halogenated polyolefin, polysaccharide, vinylic polymer, polyphosphazene, polysiloxane, polypropylene (PP), poly(tetrafluroethylene) (PTFE), poly(methymethacrylate), polyetheretherketone (PEEK) and the like. Examples of fibers include plastic, carbon or glass fibers. In some embodiments, the fibers are processed (e.g., injection molded or extruded) with an elastomer to encapsulate the fibers, thereby providing protection from tissue ingrowth and improving torsional and flexural stiffness.

Contained within the spring housing 103 is a spring 106 that actuates the return of the appendage or digit to its resting position when the antagonist muscle group relaxes. When the antagonist muscle group is contracted, the spring 106 extends to allow the normal antagonist motion of the joint. In some embodiments, the spring 106 is manufactured from a durable metal such as stainless steel, titanium and titanium alloys, tantalum and tantalum alloys, cobalt-chromium alloys, zirconium and zirconium alloys, nickel and nickel alloys. In a further embodiment, the durable metal is titanium. In another further embodiment, the durable metal is an alloy comprising titanium. In a still further embodiment, the alloy comprises titanium and nickel. In a yet still further embodiment, the alloy comprising titanium and nickel is nitinol. In other embodiments, the durable metal comprises tantalum or cobalt-chromium alloy.

Still referring to FIGS. 1A-C, in some embodiments, the proximal end of the spring 106 is attached to a sealing cap 107 that seals the proximal end of the spring housing 103 and retains the spring 106 within the spring housing 103. In some embodiments, the distal end of the spring 106 attaches to a rod 108 that is partially within the spring housing 103. Contact between the rod 108 and the walls of the spring housing 103 is snug enough to essentially seal the distal end of the spring housing 103, but not tight enough to impede the travel of the rod 108. In this configuration, the contraction of the antagonist muscle group pulls the rod 108 outward from the spring housing 103, but not far enough to completely pull the rod 108 out of the spring housing 103 or break the essential seal the distal end of the spring housing 103. When the antagonist muscle group relaxes, the spring 106 pulls the rod 108 in the proximal direction, back into the spring housing 103.

The distal end of the rod 108 comprises a hole 109 for attachment. In some embodiments, the tendon(s) of interest is threaded through the hole 109 and sutured back to itself by any tendon weave technique known in the art. In other embodiments, a tether is passed through the hole 109 and the tether is sutured to the tendon by any technique known in the art. In still other embodiments, a tether is passed through the hole 109 and the tether is secured at its other end to a plate, screw or anchor embedded in or attached to a bone of the appendage, limb or digit of interest. In particular embodiments, the tether is composed of a durable metal or a biocompatible, non-degradable, preferably non-elastic polymeric material. In some embodiments, the thickness of the rod 108 in the region of the hole 109 is the same as the thickness of the rest of the rod 108. In other embodiments, the region of the hole 109 is thinner than the rest of the rod 108 in order to make tendon or tether connection easier.

In some embodiments, the cross-sectional profile of the spring housing 103 is a circle or a circle with a flattened side. In other embodiments, the cross-sectional profile of the spring housing 103 is an oval or an oval with a flattened side. Depending on the shape of the spring housing 103, the spring 106, rod 108 and sealing cap 107 are made in a corresponding shape to fit into the spring housing 103. In one embodiment, the spring housing 103 has a cross-sectional profile of an oval with a flattened side. In this configuration, the flattened sides of the oval spring housing 103 face toward and away from the long bone the device 100 is mounted upon, in order to lower the profile of the device 100 under the skin.

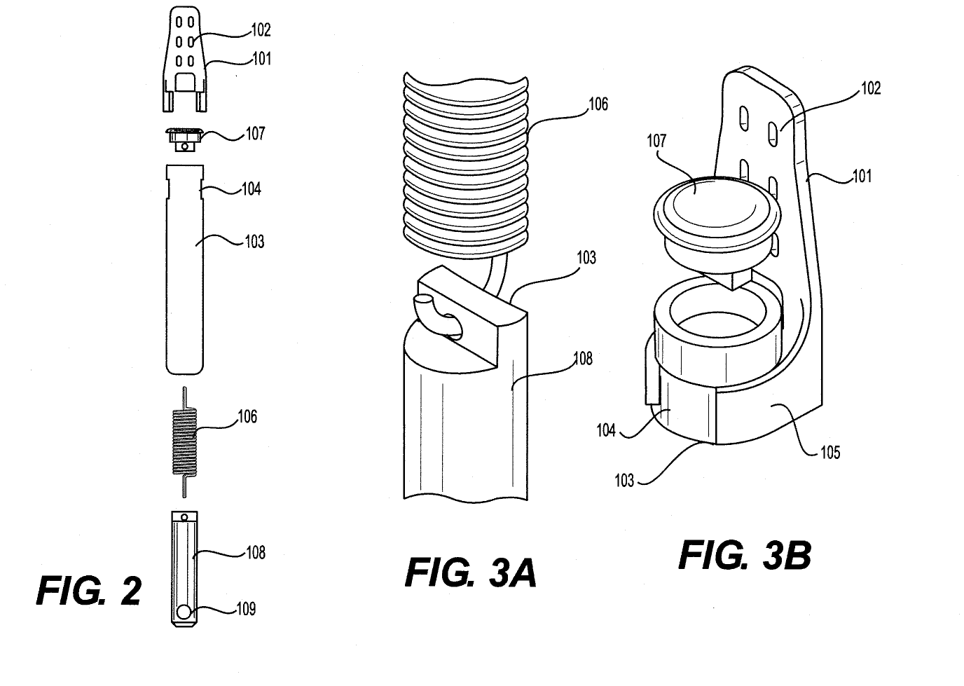

FIG. 2 shows an exploded view of the individual components of the embodiment of the device 100 depicted in FIGS. 1A-C.

FIG. 3A shows a detailed view of an exemplary, but non-limiting, connection between the spring 106 and rod 108 within the spring housing 103. FIG. 3B shows a detailed view of an example of how the groove 104 of the spring housing 103 may be retained by the brackets 105 of the fixation plate 101.

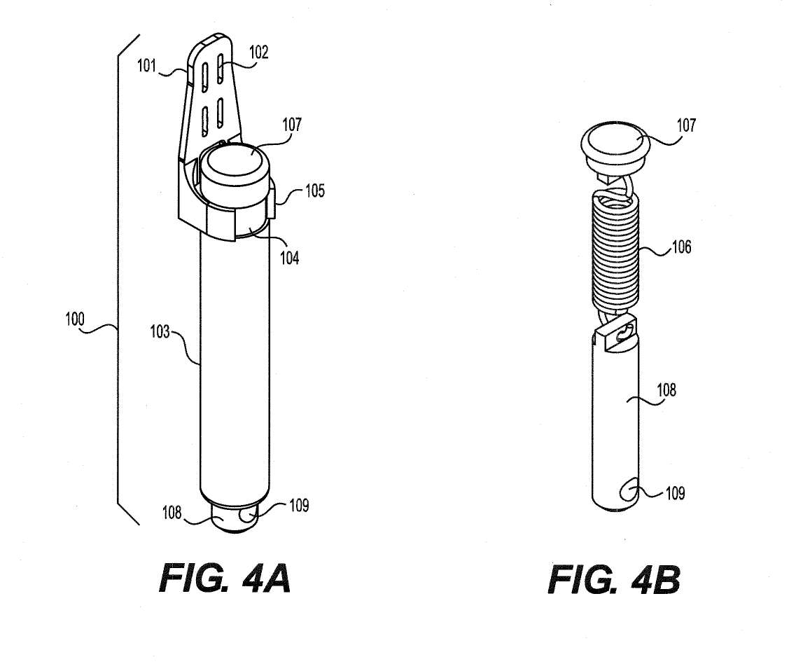

Turning now to FIGS. 4A-B, 3-dimensional perspective views of the external and internal components, respectively, of the device 100 shown in FIG. 1. The overall length of the device 100 is generally dependent upon the application for which it is being used. For example, embodiments of the device 100 being used in the lower leg for the treatment of foot drop, in general will be larger than embodiments of the device 100 being used in the forearm for the extension of the fingers.

In some embodiments, the overall length of the device 100 in a resting state, from the proximal surface of the sealing cap 107 to the exposed distal surface of the rod 108, is between about 10-400 mm. In a further embodiment, said overall length of the device 100 in a resting state is about 10-20 mm, 10-50 mm, 10-100 mm, 10-150 mm, 10-200 mm, 10-250 mm, 10-300 mm, 10-350 mm, 10-400 mm, 20-50 mm, 20-100 mm, 20-150 mm, 20-200 mm, 20-250 mm, 20-300 mm, 20-350 mm, 20-400 mm, 50-100 mm, 50-150 mm, 50-200 mm, 50-250 mm, 50-300 mm, 50-350 mm, 50-400 mm, 100-150 mm, 100-200 mm, 100-250 mm, 100-300 mm, 100-350 mm, 100-400 mm, 150-200 mm, 150-250 mm, 150-300 mm, 150-350 mm, 150-400 mm, 200-250 mm, 200-300 mm, 200-350 mm, 200-400 mm, 250-300 mm, 250-350 mm, 250-400 mm, 300-350 mm, 300-400 mm or 350-400 mm.

In some embodiments, the device 100 is designed for use in feet or legs, and has an overall length between about 12.5 mm and about 250 mm in a resting state. In a further related embodiment, the overall length of the device 100 in a resting state is between about 90 mm and about 160 mm. In a further related embodiment, the overall length of the device 100 in a resting state is between about 105 mm and about 145 mm. In a still further related embodiment, the overall length of the device 100 in a resting state is about 125 mm. In some embodiments, the device 100 have a cross section dimension of 5-100 mm, 8-62.5 mm or 12.5-50 mm in all directions.

In other related embodiments, the device 100 is designed for use in arms or hands, and has an overall length of between about 25 mm and about 150 mm the device 100 in a resting state. In a further related embodiment, said overall length of the device 100 in a resting state is between about 40 mm and about 80 mm. In a further related embodiment, said overall length of the device 100 in a resting state is between about 50 mm and about 70 mm. In a still further related embodiment, said overall length of the device 100 in a resting state is about 60 mm. In some embodiments, the device 100 have a cross sectional dimensions of 5-40 mm, 8-30 mm or 12.5-25 mm.

In the above-described embodiments, the device 100 is stretchable to a stretched length at is up to 120%, 130%, 140%, 150%, 160%, 170%, 180%, 190% or 200% of the resting length.

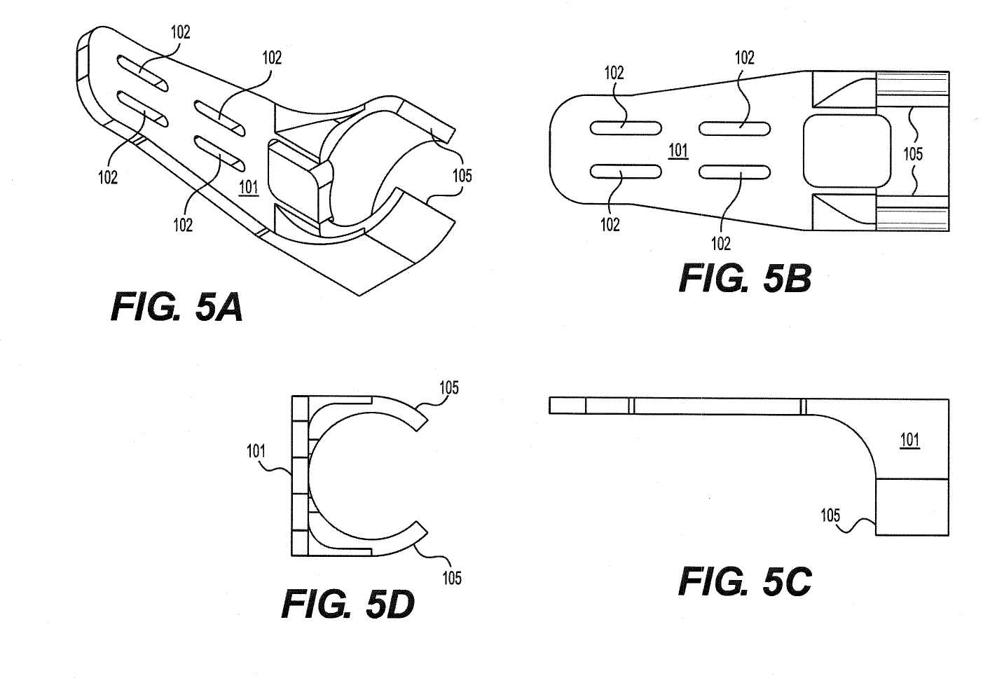

FIGS. 5A-D provide a detailed view of the fixation plate 101 for the device 100 as it is depicted in FIG. 4A. As stated above, the number of holes 102 for the insertion of fasteners to secure the fixation plate 101 to a bone is variable and dependent upon the application of the device 100. Depicted here is an exemplary embodiment having four holes for fasteners.

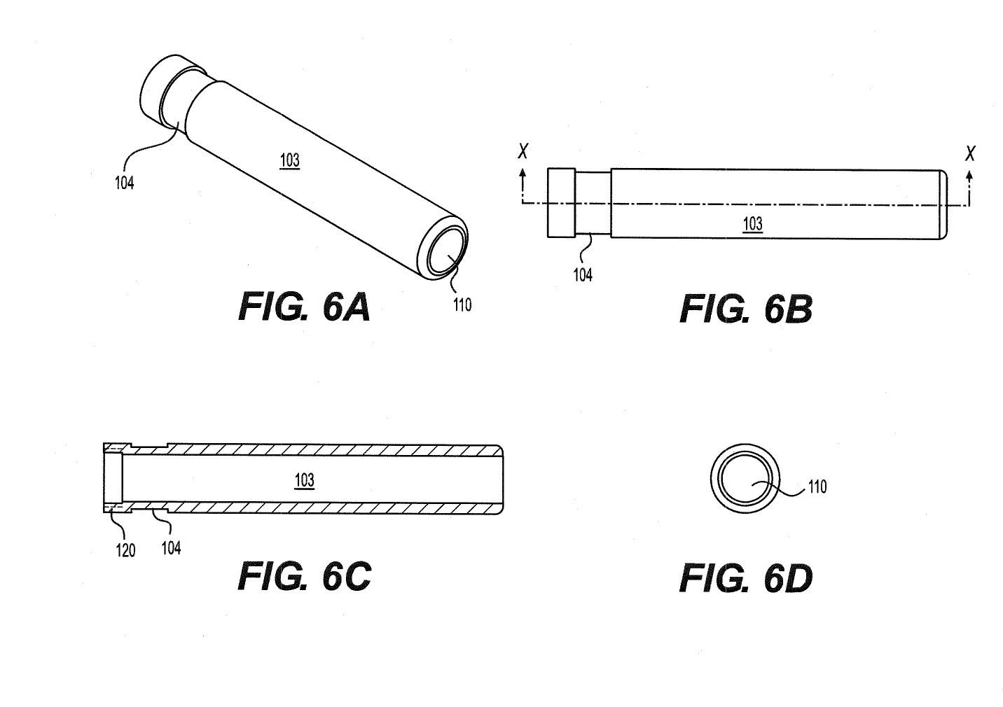

In FIGS. 6A-D, an exemplary embodiment of the spring housing 103 of the device 100 shown in FIG. 1. FIG. 6A is a three-dimensional perspective view of the spring housing 103 with the lumen 110 of the housing 103 visible at the distal end, from where the rod 108 protrudes (FIG. 4A, for example). FIG. 6B is a lateral view of the spring housing 103 showing the groove 104 that interfaces with the bracket 105 (FIG. 4A, for example) of the fixation plate 101 to secure the device 100 to the bone. FIG. 6C is a lateral-sectional view of the spring housing 103 at line X-X in FIG. 6B. In some embodiments, the proximal end 120 of the spring housing 103 is machine-threaded so that the cap 107 can be screwed into the spring housing 103. In other embodiments, the proximal end 120 of the spring housing 103 is smooth, and the sealing cap 107 is secured to the proximal end 120 of the spring housing 103 with adhesive or welding. FIG. 6D is a view looking at the distal end of the spring housing 103, showing the center lumen 110.

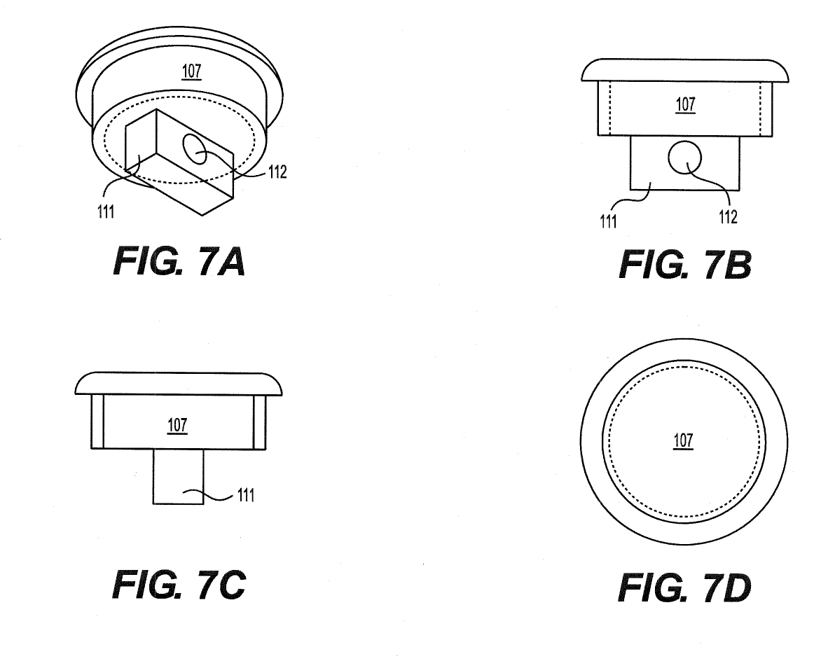

FIGS. 7A-D show detailed views of the sealing cap 107 that inserts into the proximal end of the spring housing 103 of the device 100. FIG. 7A is a three-dimensional perspective view of the sealing cap 107 with the tab 111 comprising a hole 112 for the insertion of the proximal end of the spring 106 (FIG. 4B, for example). FIGS. 7B and C are lateral views of the sealing cap 107, turned 90 degrees to one another. FIG. 7D is a view of the proximal surface of the sealing cap 107.

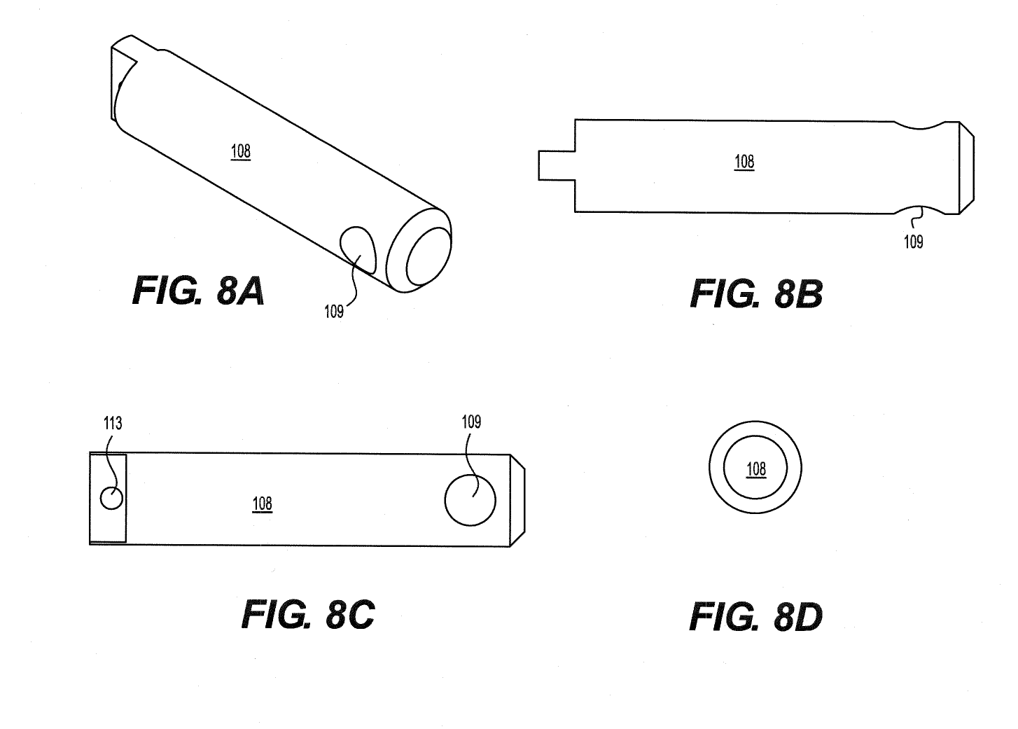

FIGS. 8A-D show detailed views of the rod 108 that protrudes through, and seals, the distal end of the spring housing 103 of the device 100. FIG. 8A is a three-dimensional perspective view of the rod 108 with the tab 114 comprising a hole 113 for the insertion of the distal end of the spring 106 (FIG. 4B, for example). FIGS. 8B and C are lateral views of the rod 108, turned 90 degrees to one another. FIG. 8D is a view of the distal surface of the rod 108.

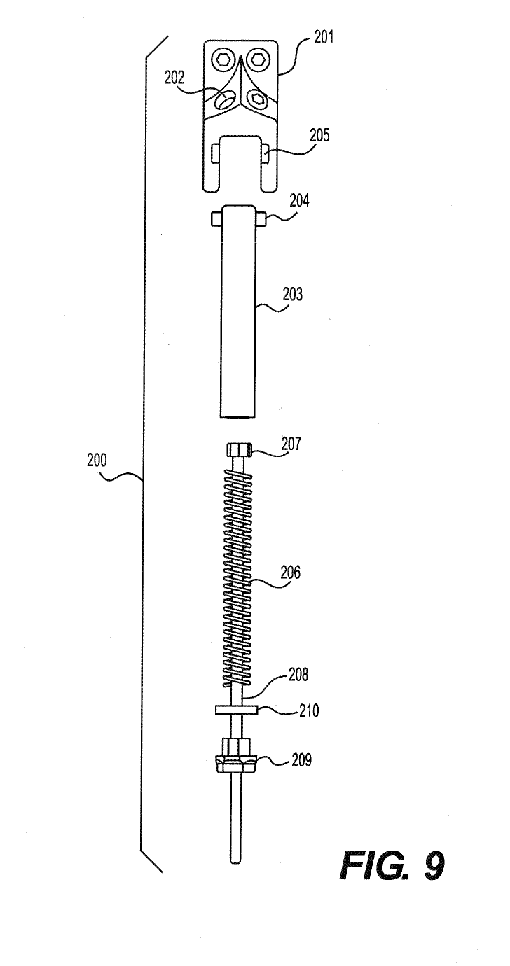

FIG. 9 shows an exploded view of another embodiment of the device of the present application. In this device 200, the spring 206 is compressed when the antagonistic muscle group contracts, pulling the distal end of the tether 208 outward from the spring housing 203 and extending the total length of the embodiment. When the antagonistic muscle group relaxes, the spring 206 expands, pushing against a knob 207 that is attached to the proximal end of the tether 208, pulling the tether 208 back inward to the spring housing 203, thus actuating the return of the appendage, limb or digit to the resting position. This device 200 also depicts another embodiment for attachment of the spring housing 203 to the fixation plate 201. In this embodiment, the proximal end of the spring housing 203 is sealed and comprises pegs 204 that interface with notches or grooves 205 in the fixation plate 201, thereby securing the spring housing 203 to the fixation plate 201. The tether 208 passes through an opening in a sealing cap 209 at the distal end of the spring housing 203. In some embodiments, the device 200 further comprises a crushable washer 210 to enhance the seal between the sealing cap 209 and the spring housing 203. Contact between the tether 208 and the sides of the opening in the sealing cap 209 is snug enough to essentially seal the distal end of the spring housing 203, but not tight enough to impede the travel of the tether 208.

Still referring to FIG. 9, the fixation plate 201 is secured to a long bone by the insertion of a fastening device through at least one hole 202 in the plate 201. In some embodiments, the fixation plate 201 has 1, 2, 3, 4, 5, 6, 7, 8, 9, 10 or more holes 202. In a particular embodiment, the fixation plate 201 has 4-8 holes. The number of holes and fastening devices used is dependent upon the location and size of the device, as well as the load placed upon the device by the movement of the appendage or digit. In some ambodiments, the plate has a 3rd dimension to it, allowing screws to be placed at an angle for better fixation of the plate to the bone. In some embodiments, a fastening device is inserted through each of the holes 202 in the plate 201. In other embodiments, at least one hole 202 does not have a fastening device inserted there through. In particular embodiments, the pattern of which holes have an inserted fastening device and which holes do not may be dependent upon whether the device is employed on a left or right limb of the patient's body.

Again referring to FIG. 9, the tether 208 is sutured to a tendon of interest by any technique known in the art. In still other embodiments, the tether is secured at its distal end to a plate, screw or anchor embedded in or attached to a bone of the appendage, limb or digit of interest.

The fixation plate 201, spring housing 203, flanged cap 204, spring 206, anchor 207, tether 208 and sealing cap 209 can be made from any suitable medically acceptable material, such as metal, biocompatible non-degradable polymeric materials, ceramic and fiber materials. Examples of suitable metals include, but are not limited to, stainless steel, titanium and titanium alloys, tantalum and tantalum alloys, cobalt-chromium alloys, zirconium and zirconium alloys, nickel and nickel alloys. Examples of biocompatible non-degradable polymeric materials include, but are not limited to, polyester (e.g., Dacron®), polyethylene, polyaramid, poly-paraphenylene terephthalamide (e.g., Kevlar®), polyethylene terephthalate, acrylic polymers, methacrylic polymers, polyurethane, polyurea, polyolefin, halogenated polyolefin, polysaccharide, vinylic polymer, polyphosphazene, polysiloxane, polypropylene (PP), poly(tetrafluroethylene) (PTFE), poly(methymethacrylate), polyetheretherketone (PEEK). Examples of fiber materials, include plastic, carbon or glass fibers.

In some embodiments, the device 200 comprises a single spring housing 203. In other embodiments, dependent upon the application, the device 200 comprises two spring housings 203, for example, when each spring housing comprises a spring that is functionally attached to a separate tendon, or are functionally tethered to opposite sides of the foot to aid in foot leveling. In still other embodiments, dependent upon the application, the device 200 comprises 2, 3, 4 or 5 spring housings 203, for example, when each spring housing 203 comprises a spring that is functionally attached to a separate digit of the hand, for example to allow the separate extension of each connected digit.

In some embodiments, the cross-sectional profile of the spring housing 203 is circular. In other embodiments, the cross-sectional profile of the spring housing 203 is a flattened oval, with the spring 206 and sealing cap 209 being in a corresponding flattened oval shape. In such a configuration, the flattened sides of the oval spring housing 203 face toward and away from the long bone the device 200 is mounted upon, in order to lower the profile of the device under the skin.

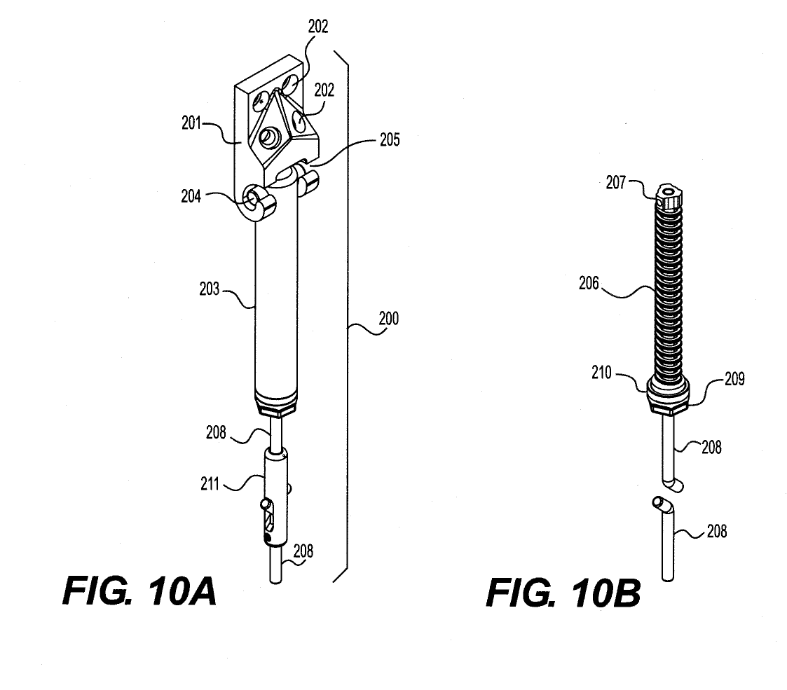

FIGS. 10A-B are 3-dimensional perspective views of the external and internal components, respectively, of the device 200 shown in FIG. 9. The overall length of the device 200 is generally dependent upon the application for which it is being used. For example, embodiments of the device 200 being used in the lower leg for the treatment of foot drop, in general will be larger than embodiments of the device 200 being used in the forearm for the extension of the fingers.

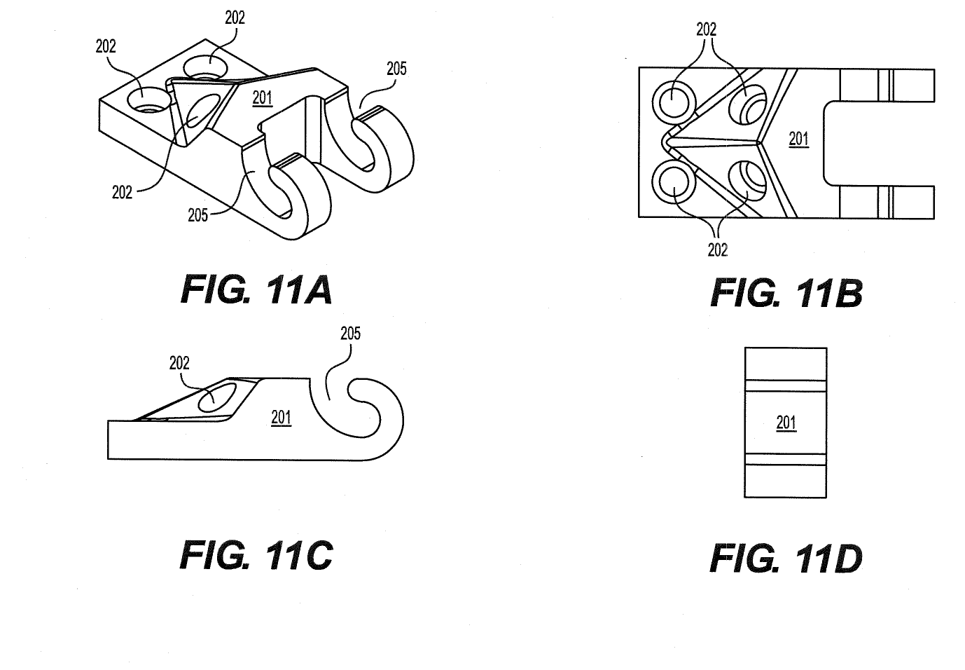

FIGS. 11A-D provide a detailed view of the fixation plate 201 for the device 200 as it is depicted in FIG. 9. As stated above, the number of holes 202 for the insertion of fasteners to secure the fixation plate 201 to a bone is variable and dependent upon the application of the device 200. Depicted here is an exemplary embodiment having four holes for fasteners.

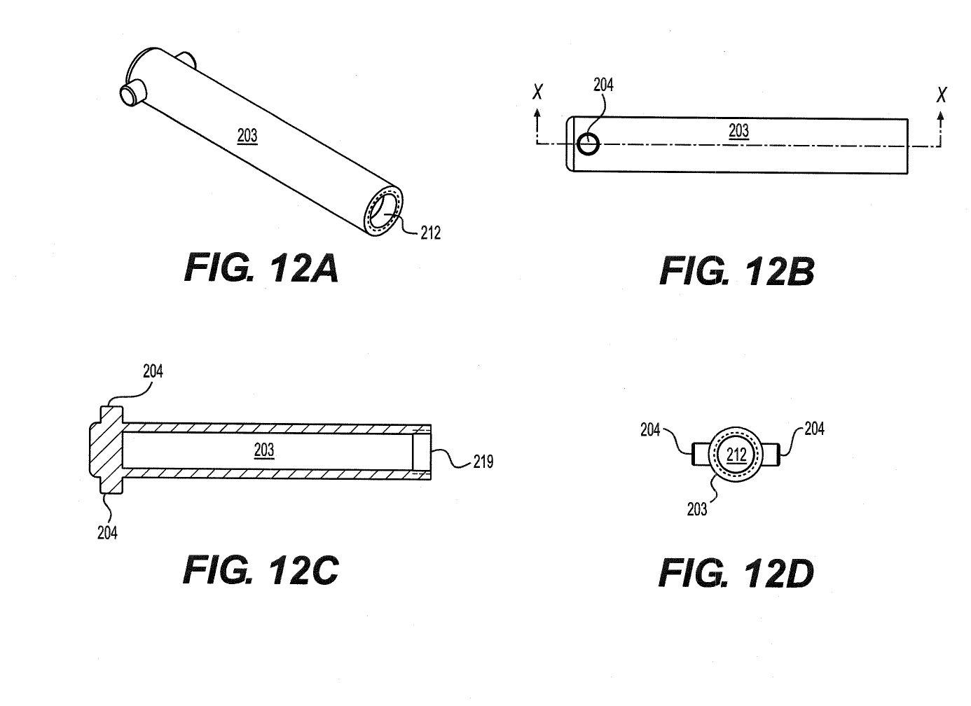

In FIGS. 12A-D, an exemplary embodiment of the spring housing 203 of the device 200 shown in FIG. 9. FIG. 12A is a three-dimensional perspective view of the spring housing 203 with the lumen 212 of the housing 203 visible at the distal end, where the distal sealing cap 209 attaches (FIG. 10A, for example). FIG. 12B is a lateral view of the spring housing 203 showing the pegs 204 that interface with the slots 205 (FIG. 10A, for example) of the fixation plate 201 to secure the device 200 to the bone. FIG. 12C is a lateral-sectional view of the spring housing 203 at line X-X in FIG. 12B. In some embodiments, the distal end 219 of the spring housing 203 is machine-threaded so that the cap 209 can be screwed into the spring housing 203. In other embodiments, the distal end 219 of the spring housing 203 is smooth, and the cap 209 is secured to the distal end 219 of the spring housing 203 with adhesive or welding. FIG. 12D is a view looking at the distal end of the spring housing 203, showing the center lumen 212 and the pegs 204.

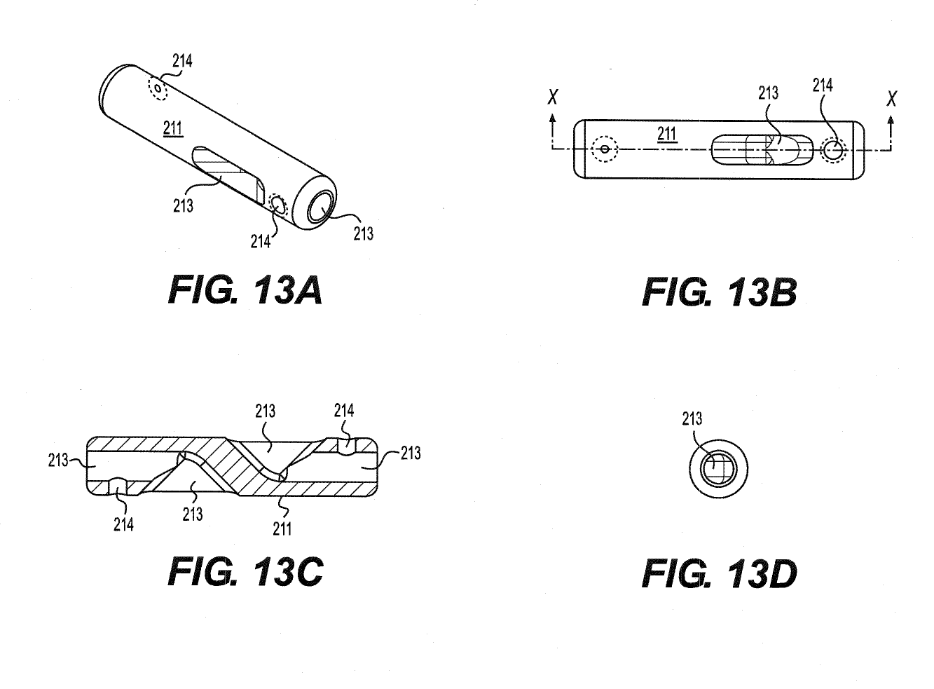

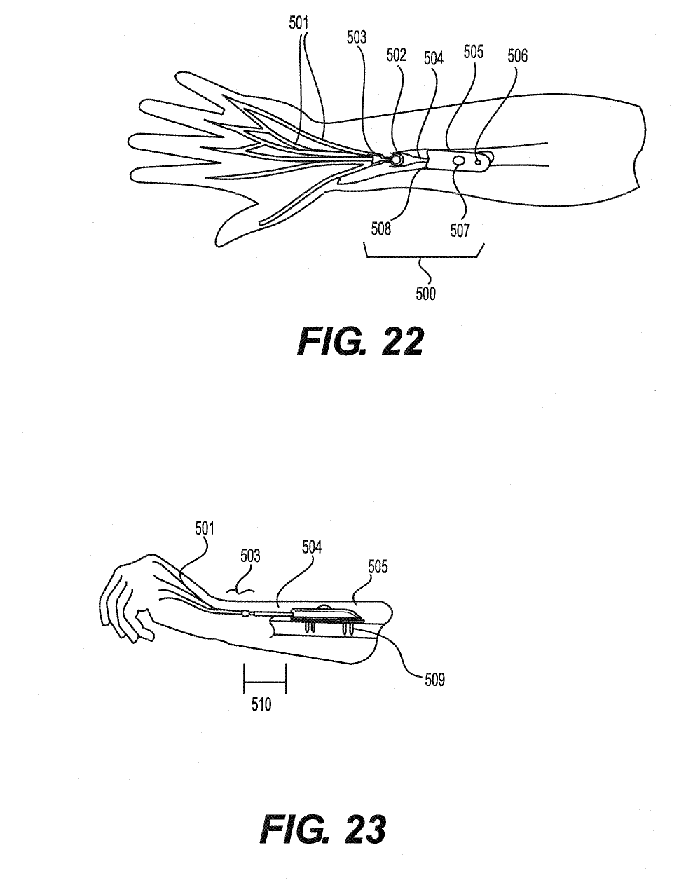

FIGS. 13A-D show a buckle 211 that optionally attaches to the tether 208 of the device 200 in order to adjust the length or tension of the tether 208 during implantation of the device 200. In some embodiments, the buckle 211 can also be used in conjunction with the elastic tether of the device 400 depicted in FIGS. 21 and 22. FIG. 13A is a three-dimensional perspective view of the buckle 211 with one of the locking channels 213 of the buckle 211 visible at the end, with an identical locking channel 213 hidden in this view. FIG. 13B is a lateral view of the buckle 211, again showing one of the locking channels 213, with the other channel hidden. FIG. 13C is a lateral-sectional view of the buckle 211 through both locking channels 213 at line X-X in FIG. 13B. Each channel is contiguous with a screw hole 214 though which a screw is applied and secured against the tether 208 to compress and secure the tether 208 in the locking channel 213. FIG. 12D is a view looking at the one end of the buckle 211, showing one of the locking channels 213.

As illustrated in FIGS. 10A-B, the distal end of a section of tether 208 protruding through the cap 209 on the distal end of the spring housing 203 is fed through the locking channel 213 at the proximal end of the buckle 211. Additionally, the proximal end of a tether 208′ that is distally connected to a tendon or bone of the limb, appendage or digit of interest is threaded through the locking channel 213 at the distal end of the buckle 211. Once the practitioner has determined that the amount of tension on the tether 208 is correct, the ends of the tethers 208 and 208′ are secured in place in the locking channels 213 with fasteners through the screw holes 214.

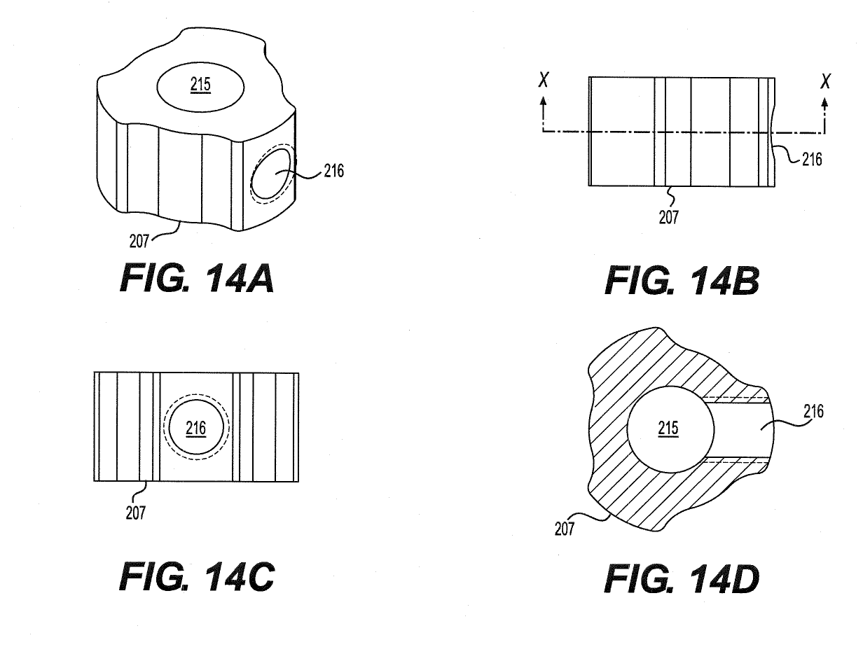

FIGS. 14A-D show the anchor 207 that secures the proximal end of the tether 208 through the center of the spring 206 in the lumen of the spring housing 203. FIG. 14A is a three-dimensional perspective view of the anchor 207 with the channel 215 that the tether 208 passes through visible. The anchor 207 comprises a screw hole 216 through which a fastener is inserted to compress and secure the tether 208 in the anchor 207. FIGS. 14B and C are lateral views of the anchor, turned 90 degrees to one another. FIG. 14D is a cross-sectional view of the anchor 207 through the channel 215 and screw hole 216 at line X-X in FIG. 14B.

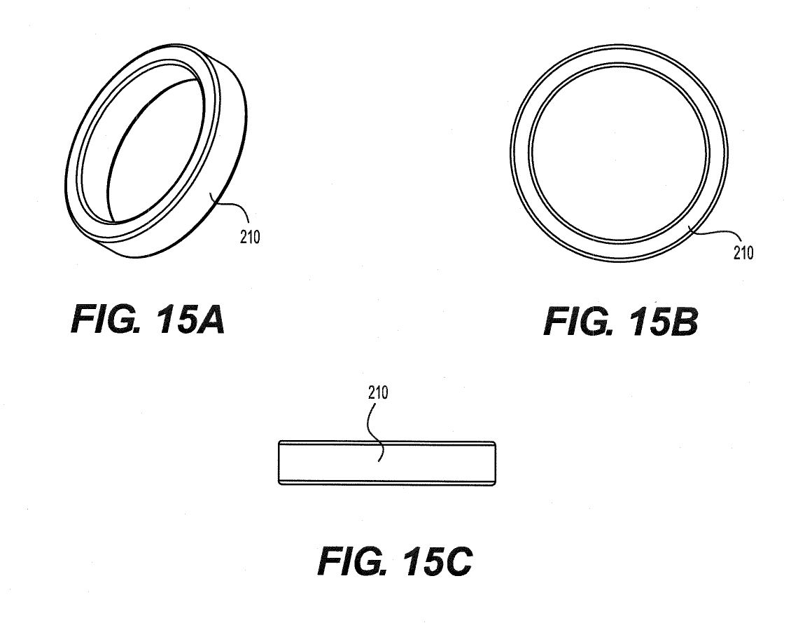

FIGS. 15A-C are perspective views of the gasket 210 that is compressed between the spring housing 203 and cap 209 (FIG. 10A) in order to seal fluids out of the spring housing 203.

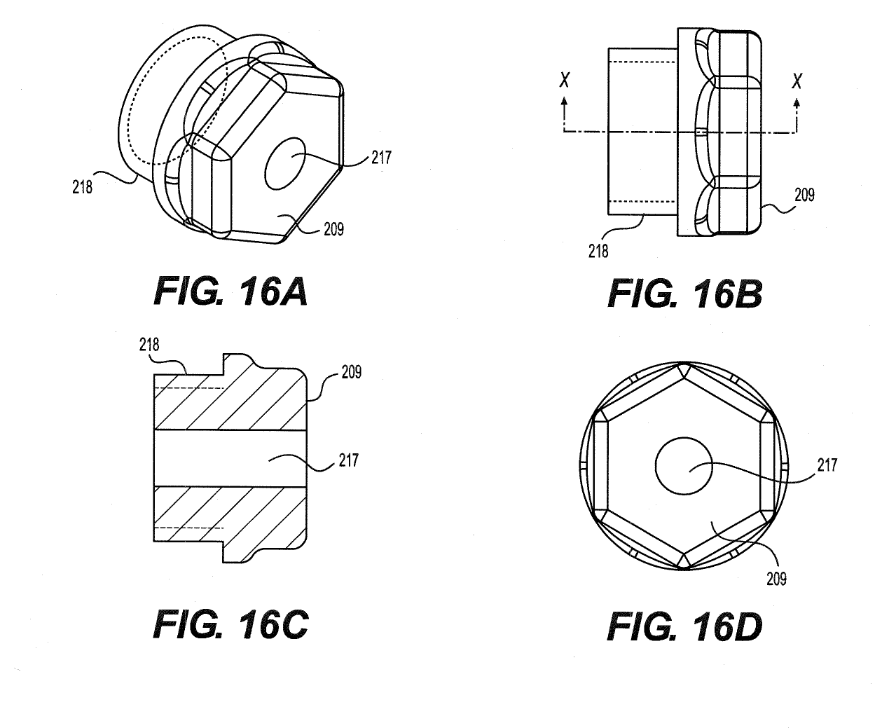

With attention now on FIGS. 16A-D, perspective views of a cap 209 for the spring housing 203 of the device 200 are shown. FIG. 16A is a three-dimensional perspective view of the cap 209 with the channel 219 that the tether 208 passes through. FIG. 16B is a lateral view of the cap 209 and FIG. 16C is a cross-sectional view of the cap 209 through the channel 217 at line X-X in FIG. 16B. In some embodiments, the proximal end 218 of the cap 209 is machine-threaded so that the cap 209 can be screwed into the spring housing 203. In other embodiments, the proximal end 218 of the cap 209 is smooth, and the cap 209 is secured to the distal end 219 of the spring housing 203 with adhesive or welding. FIG. 16D is a view looking at the distal end of the cap 209, showing the center channel 217 for the tether 208 (see FIGS. 10A-B).

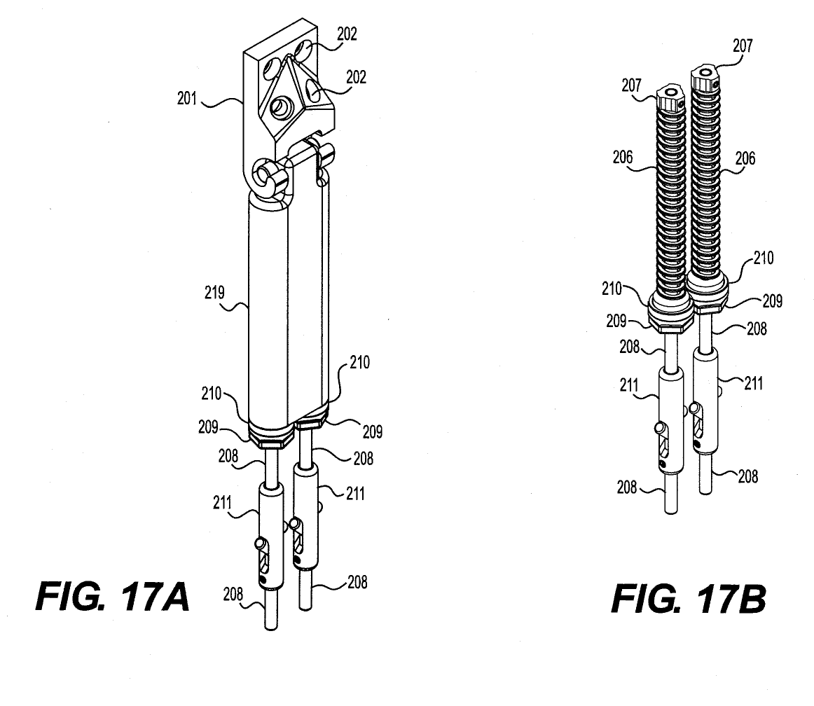

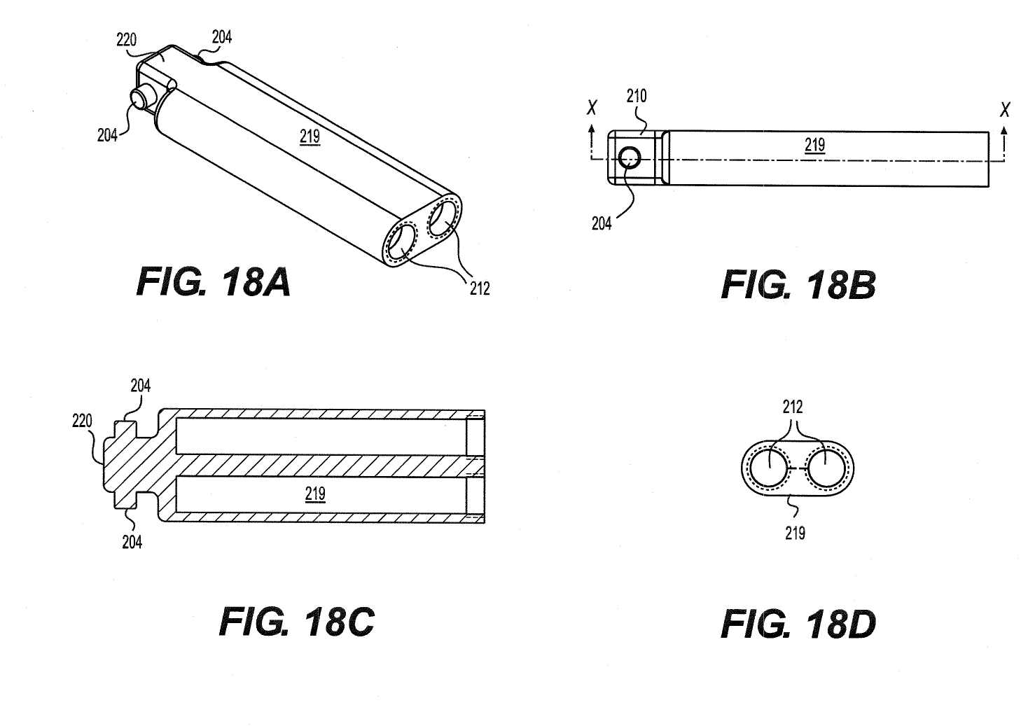

As discussed above, in some embodiments, the device 200 comprises two or more spring housings 203. In other embodiments, the device 200 comprises a single housing with two or more cylinder-shaped chambers. FIGS. 17A-B and 18A-D show an embodiment of the device 200 having a dual-chambered spring housing 219. Such an embodiment is useful, for example, for functionally connecting separate tethers 208 to either side of the foot to aid in foot leveling. The multi-chambered embodiment 219 also has the advantage of all spring mechanisms sharing a single fixation plate 201, simplifying the implantation procedure and requiring no more fasteners inserted into the bone than required for a single chambered device. As seen in FIGS. 17A and 18A-C, in some multi-chambered 219 device 200 embodiments, the pegs 204 are located on an extended proximal tab 220 in order to connect to the fixation plate 201 and allow clearance of the additional chambers 212. However, all of the other functional components of the device 200 can be identical to those described above for FIGS. 9-16.

Additionally, a similar multi-chambered configuration is also contemplated for the device 100 described above in FIGS. 1-8.

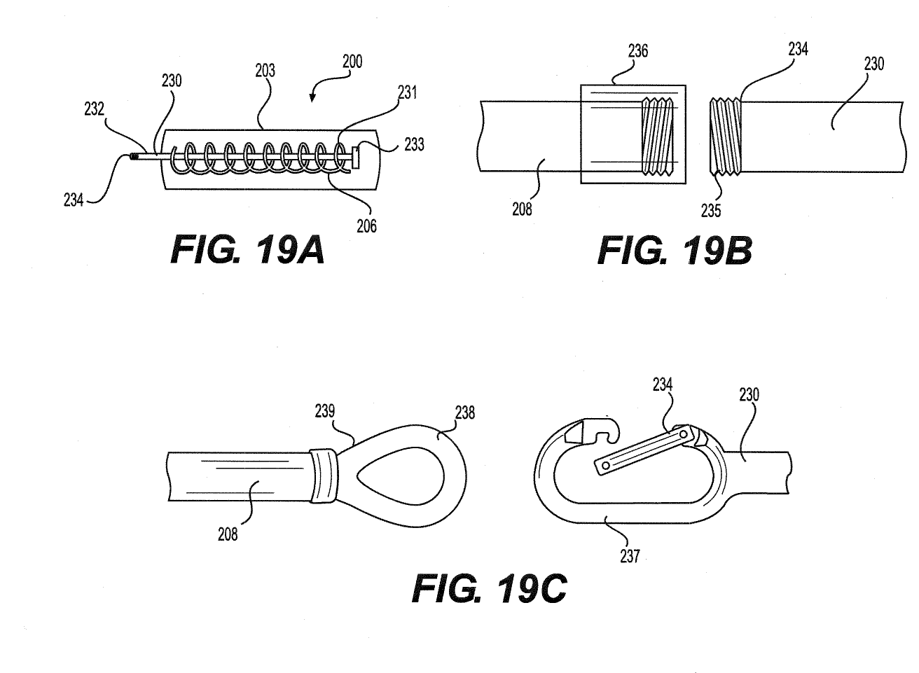

FIGS. 19A-C show another embodiment of the device 200. As shown in FIG. 29A, the spring 206 is mounted on a rod 230 having a proximal end 231 and a distal end 232. The proximal end 231 had an anchor 233 that compresses the spring 206 when the device 200 is in a stretched state. The distal end 232 contains an attachment section 234 that is removably attached to the tether 208. In this design, the tether 208 does not enter the housing 203 so as to avoid friction between the tether 208 and the housing 203 or spring 206. This design also allows for better sealing of the housing 203. FIGS. 19B and 19C show two exemplary embodiments of the attachment section 234 of the rod 230. In one embodiment, the attachment section 234 comprises raised or depressed spiral lines 235 that can be screwed onto a screw connector 236 which is attached to the tether 208 through a similar mechanism (FIG. 19B). This design allows for easy exchange or replacement of the tether 208. In another embodiment, the attachment section 234 of the rod 230 comprises a carabiner type end 237 and the tether 208 contains a loop 238 at its proximal end 239 (FIG. 19C). This design allows for easy connection or disconnection between the rod 230 and the tether 208.

### Elastic Tether Internal Dynamic Splint Devices

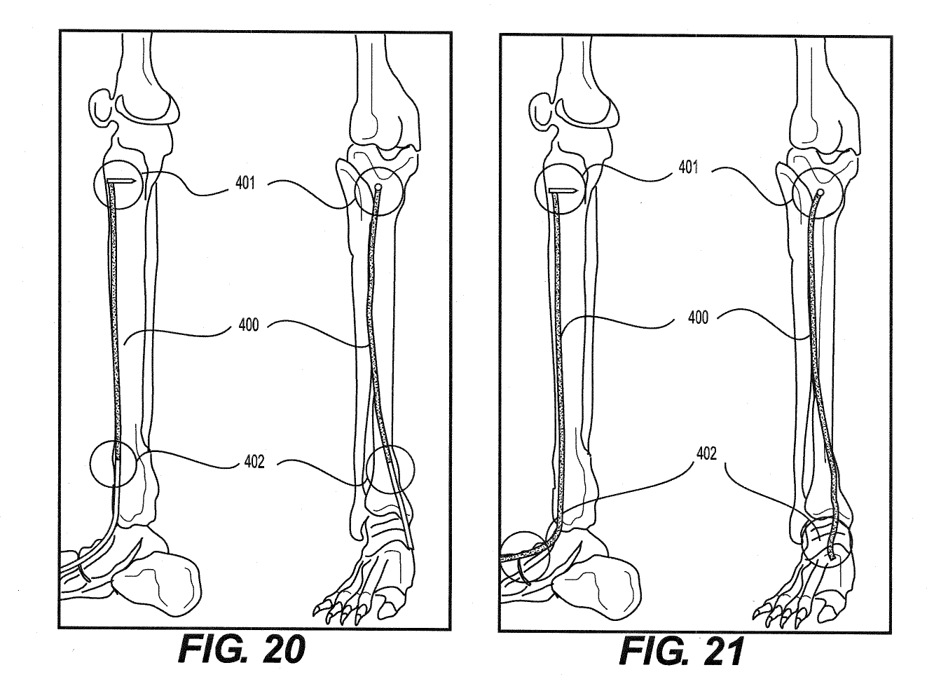

FIGS. 20 and 21 show embodiments of the device utilizing an elastic band or a bundle of elastic band 400 material rather than a spring-loaded mechanism and without the cylinder or plate of the embodiments depicted in FIGS. 1-19. The elastic band 400 can be made of any biocompatible elastic material, such as, but not limited to silicon, biocompatible non-degradable polymers such as polyester, polyethylene, or Kevlar, and metals. The elastic band 400 may comprise one or more elastic bands. In some embodiments, the elastic band 400 contains a single elastic band with a desired stiffness or elasticity. In other embodiments, the elastic band 400 contains two or more elastic bands that form a bundle. The multiple bands in the bundle may have the same or different stiffness. In some embodiments, the elastic band in elastic band 400 further comprises a braid of multiple strings. The elasticity of the elastic band is determined by the elasticity of the individual strings and the pattern the multiple strings are braided.

The elastic band 400 is anchored at its proximal end 401 to the long bone. In some embodiments, the elastic band 400 is anchored at its proximal end 401 to the long bone with a suture anchor, a plate, a screw anchor, or a bio-compression screw. In still other embodiments, the elastic band 400 is anchored at its proximal end 401 to the long bone with a bone screw made of a durable metal. In a further embodiment, the durable metal is stainless steel, titanium, tantalum, nickel, gold, cobalt, chromium or alloys thereof. In one embodiment, the durable metal is nitinol or a cobalt-chromium alloy.

In FIG. 20, the distal end 402 of the elastic band 400 is sutured to a tendon of interest that is attached to the appendage by any effective known suture technique. In some embodiments, multiple tendons are sutured to the elastic band 400. In other embodiments, multiple elastic band 400 are anchored 401 to the same long bone at their proximal ends and each sutured at their distal ends 402 to individual tendons, for example on the posterior surface of the hand, being sutured to the tendons on each of the fingers.

In FIG. 21, the distal end 402 of the elastic band 400 is directly attached to a bone of the appendage, limb or digit of interest using a fastening device. In some embodiments, the fastening device is a bio-compression screw, an adjustable locking system, or a bone screw made of a durable metal. In a further embodiment, the durable metal is stainless steel, titanium, tantalum, nickel, gold, cobalt, chromium or alloys thereof. In one embodiment, the durable metal is nitinol or a cobalt-chromium alloy. In still another embodiment, the fastening device is a plate that, in turn, is attached to a bone of the appendage, limb or digit of interest using appropriate fastening device(s). In some embodiments, multiple elastic bands 400 are anchored to the same long bone at their proximal ends 401 and each attached at their distal ends 402 to individual fastening devices, for example on the medial and anterior sides of the foot so that leveling of the foot can be adjusted.

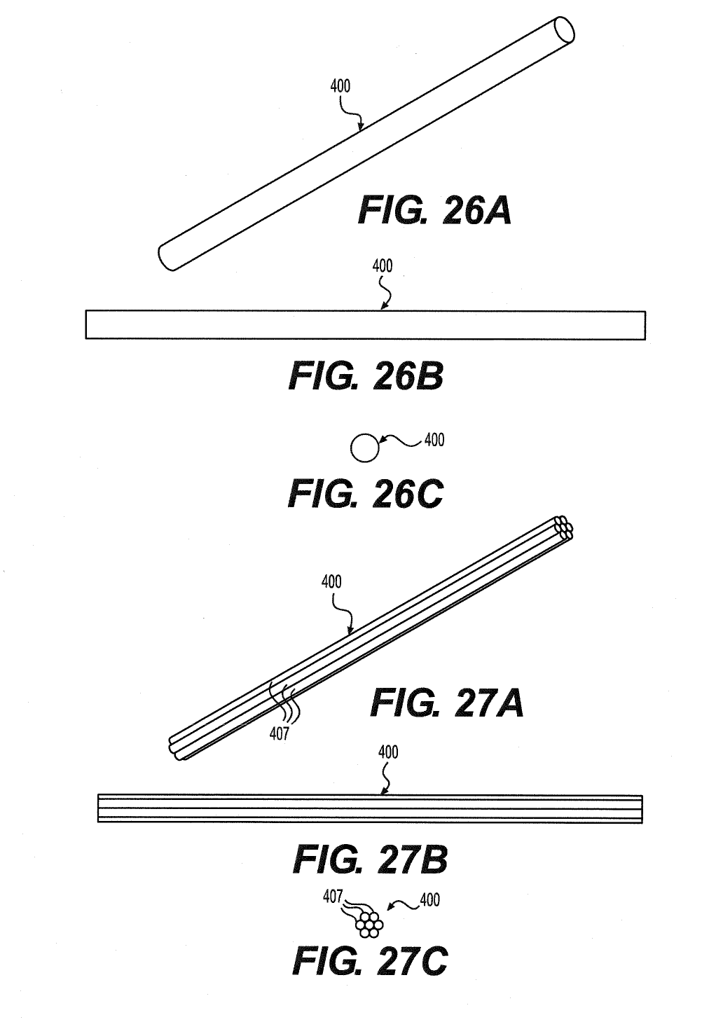

FIGS. 26A-C show an embodiment of an elastic tether 400. In this embodiment, the elastic band 400 consists of a single elastic component 403. FIGS. 26A-C show the 3-dimensioanl view, the side view and the cross-sectional view, respectively, of the elastic band 400.

FIGS. 27A-C show another embodiment of an elastic tether 400. In this embodiment, the elastic band 400 consists of a bundle of elastic components 407. FIGS. 27A-C show the 3-dimensioanl view, the side view and the cross-sectional view, respectively, of the elastic band 400.

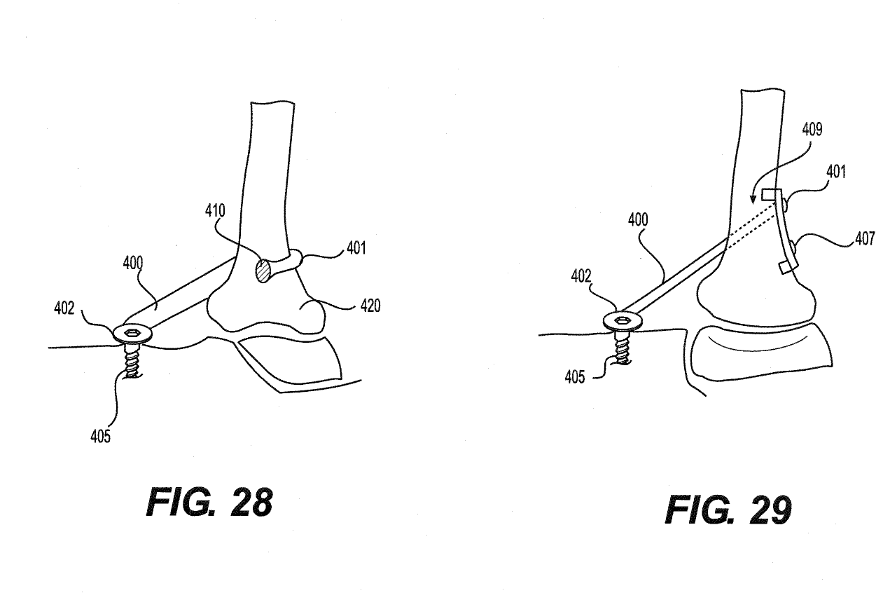

FIGS. 28 and 29 show exemplary embodiments of bone attachment of the elastic band 400 for foot drop. In one embodiment, the band 400 is wrapped around the tibia 420 medially (FIG. 28). The proximal end 401 of the elastic band 400 is attached to the tibia 420 at anchor point 410, and the distal end 402 of the elastic band 400 is mounted to a bone by a bone screw 405. In another embodiment, the elastic band 400 pass through the tibia 420 in a bone tunnel 409 (FIG. 29). The proximal end 401 of the elastic band 400 is attached to the tibia 420 at the end of bone tunnel 409 with a mounting plate 407, and the distal end 402 of the elastic band 400 is mounted to a bone by a bone screw 405. These embodiments place the elastic band 400 at a more physiologically correct angle.

### Finger Extension Internal Splint Device

FIG. 22 shows an embodiment of a finger extension internal splint device 500 having tension that is adjustable by the patient in order to adjust the grasp of the hand. In one embodiment of this device 500, the extensor tendons 501 of the fingers and thumb are commonly threaded through a keyhole 502 and each tendon 501 is woven back upon itself 503 and sutured to secure it to the device 500. The keyhole 502 is at the distal end of a piston 504, whose distal end extends into a recoil unit 505. The recoil unit 505 comprises a tension adjustment control 506, such as a screw head, which is used to adjust the tension of the device 500 during implantation. After weaving 503 the tendons 501 through the keyhole 502, tension is set by the practitioner at a level that allows the hand to be held in a neutral open position. This allows the patient to use the hand independently. The device 500 opens the hand, the patient places the hand around an object to be grasped and contracts the muscles antagonistic to the device 500, thereby overcoming the tension of the device 500 and grasping the object with enough force to control it. To release the object, the patient relaxes the antagonistic muscle and the recoil unit 505 pulls proximally on the piston 504 and the tendons 501, extending the fingers and opening the hand.

In some embodiments, the recoil unit 505 further comprises a control button 507 that can be pressed through the skin by the patient in order to select between multiple tension settings of the device, for example to adjust the speed with which the hand opens to the neutral position. The device 500 is attached to the radius via a bone anchor 508 that is secured to the bone using bone screws or any other suitable fastening device.

FIG. 23 shows a lateral view of the device 500 of FIG. 22 with the fingers flexed. The piston 504 is extended out of the recoil unit 505 and under tension by the recoil unit 505 to return in a proximal direction when the antagonistic palmar muscles are relaxed, thereby extending the fingers to an extended, neutral open position. The range of excursion 510 of the piston 504 of the device 500 should allow the joints of the fingers and the thumb to move through their functional range of motion (e.g. enough excursion that the fingers can be pulled from the position of an open hand to form a fist). FIG. 23 also shows the bone screws 509 that secure the bone anchor of the device 500 to the radius.

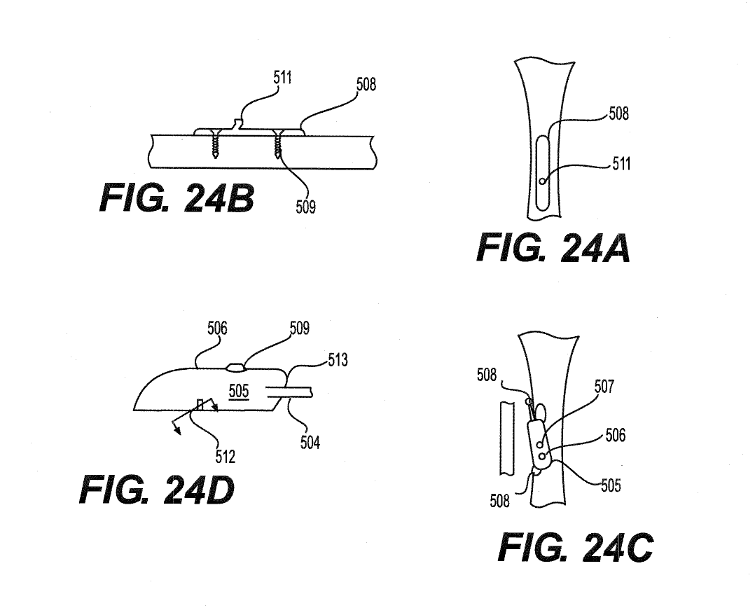

FIGS. 24A-D show the bone anchor 508 of the device 500 of FIG. 22. FIG. 24A is a top-down view of the bone anchor 508 showing the pivot 511 that the recoil unit 505 attaches to. The pivot 511 allows the recoil unit 505 to move laterally in order to compensate for changes in orientation of the tendons due to changes in the position of the wrist and hand at a given moment. FIG. 24B is a lateral view of the bone anchor 508 showing the pivot 511, as well as the bone screws 509 embedded in the radius. FIG. 24C shows the pivoting of the recoil unit 505 on the bone anchor 508.

FIG. 24D shows a simplified lateral cut-away view of the recoil unit 505 showing the relative positions of the insertion of the piston 504, the tension adjustment control 506, the transcutaneous “button” for selecting amongst preset tensioning options. 507 and the mounting point 512 for the pivot 511 of the bone anchor 508. The insertion of the piston 504 into the recoil unit 505 is surrounded by a seal 513 to prevent entry of bodily fluids into the device. The seal 513 also isolates the internal environment of the recoil uni505, allowing potentially bio-incompatible materials to be used for the recoil mechanism so as to maintain longevity of the device.

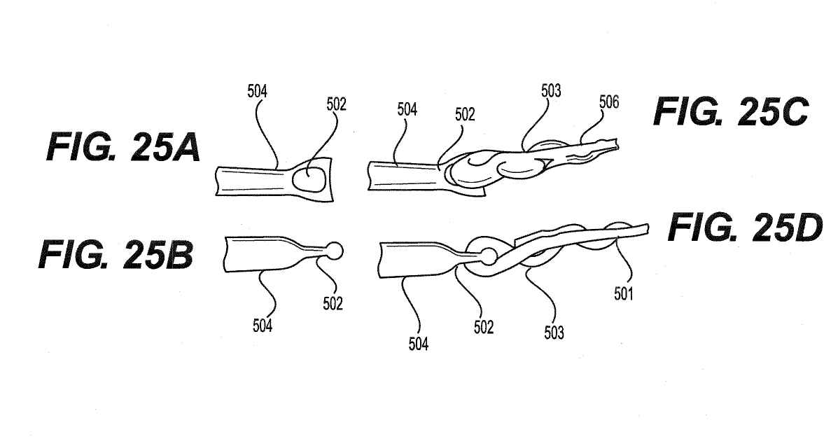

FIGS. 25A-B provide a detail view of the piston 504 and keyhole 502 from a top and lateral viewpoint, respectively. FIGS. 25C-D show an exemplary weave 503 of an extensor tendon 501 through a keyhole 502 from a top and lateral viewpoint, respectively. It should be noted that this same exemplary weave technique may also be used to secure a tendon to the rod or tether of any of the other device embodiments described in this application.

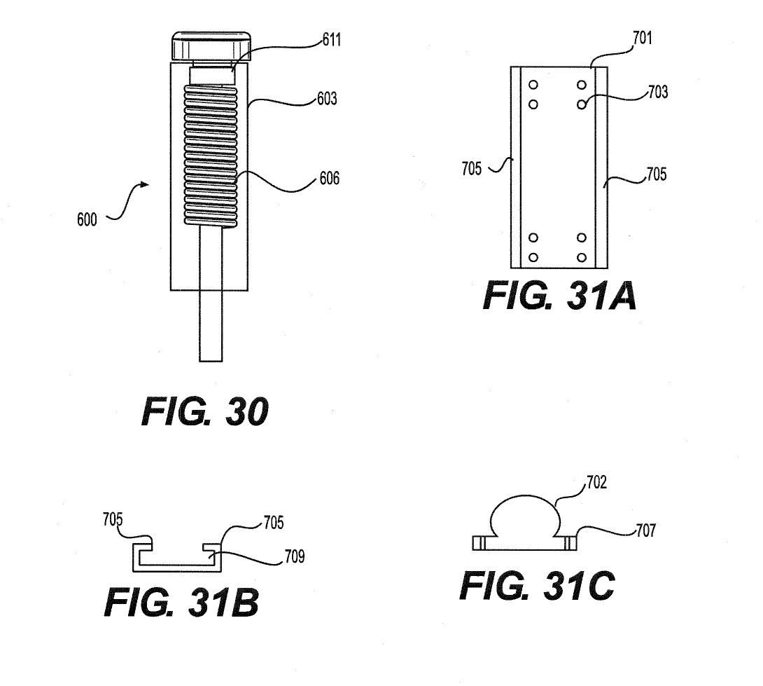

FIG. 30 shows another embodiment of a device of the present application. In this embodiment, the device 600 comprises a screw mechanism 611 at the proximal end of spring 606. The screw mechanism 611 allows the adjustment of the stiffness of the spring 606 during implantation of the device 600.

FIGS. 31A-C show another embodiment of an attachment option. In this embodiment, a large attachment plate 701 (FIGS. 31A and 31B) is secured to a bone through holes 703. The elastic body 702 is then mounted onto the attachment plate 701 through rails 705. The tails 707 of the elastic body 700 (FIG. 31C) fit into the groove 709 of the rail 705 and thus allow movement of the elastic body 702 along the rails 705 to adjust the tension or stiffness of the elastic body 702. Once a desired position (e.g., a position that provide optimal stiffness for the elastic body 702) is determined, the elastic body 700 is screwed, or otherwise fixed, to the rail plate 701 to fix the position.

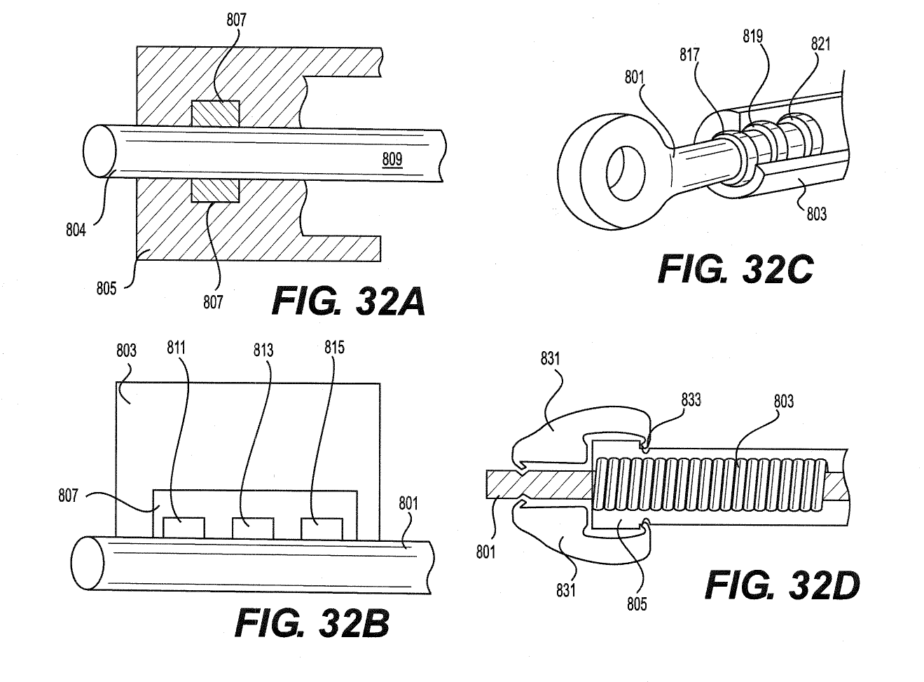

FIGS. 32A-D show some exemplary mechanisms for sealing the housing of the elastic body to prevent in-growth of tissues into the housing. As shown in FIG. 32A, the rod 801 extends through the distal end 805 of the housing 803. The interior 809 of the housing 803 is sealed to the outer environment by the seal 807. The seal 807 can be any type of seal that is capable of preventing liquid exchange between the interior 809 of the housing 803 and the outer environment. In some embodiments, the seal 807 contains multiple sealing mechanisms such as 811, 813 and 815 (FIG. 32B). Suitable sealing mechanisms include, but are not limited to, wipers, rod seals, piston seals and buffer seals. FIG. 32C shows an embodiment or a rod 801 that is sealed against the housing 803 by a wiper 817, a rod seal 819 and a buffer seal 821. FIG. 32D shows another embodiment of a sealing mechanism. In this embodiment, the rod 801 is sealed against the housing 803 by a seal 831 that wraps around the distal end 805 of the housing 803 and engages with the housing 803 in groove 833 and with the rod 801 in groove 835.

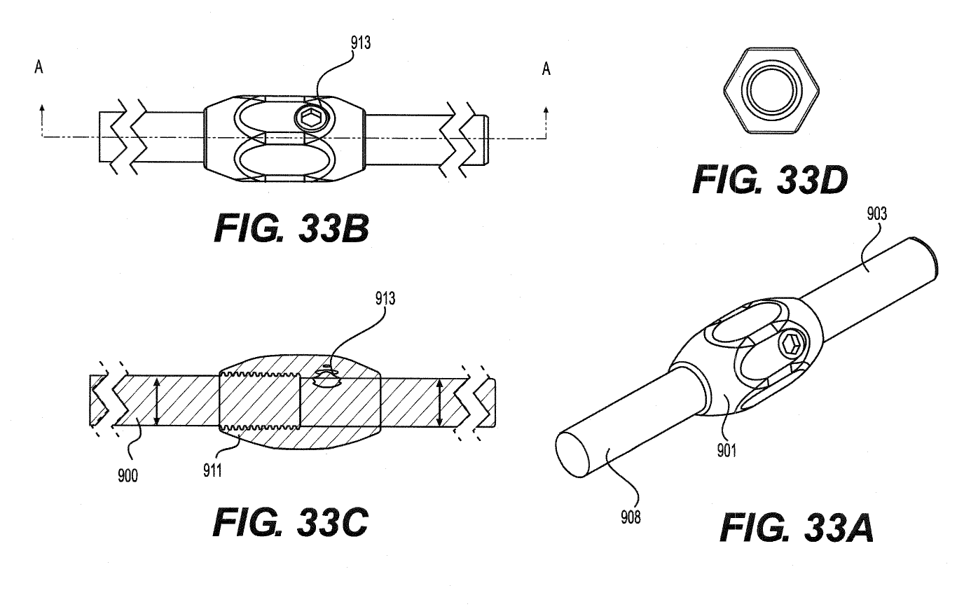

FIG. 33A-D show an embodiment of an connector 901 that connects a rod 903 to a tether 908. FIG. 33A is a 3-dimensional view of the connector 901. FIGS. 33B-33D are the side view, cross-sectional view and end view, respectively, of the connector 901. In this embodiment, the connector 901 is attached to the tether 908 through a twist-in connection 911 and is attached to the rod 903 through a screw 913. The tether 908 can be released from the connector 901 by twisting the tether 908 out of the twist-in connection 911. The rod 903 may be released from the connector 901 by un-tightening the screw 913.

### Method of Implantation

In general, the method used for surgical implantation of a device of the present application can be similar to those known in the art used for plating of a fracture or for extensor tendon repair. For example, the incision over the bone for placement of the spring housing, recoil device or attachment of the elastic tether can be performed in a location that is to one side of the ultimate location of the device so that there is no tension on the skin closure when the patient begins range of motion exercises and resumes activity.

An area of the bone is cleared, for example by subperiosteal dissection, providing adequate surface for the placement of the fixation plate (FIGS. 1-5), the attachment of the proximal end of the elastic tether (FIGS. 6-7), or the bone anchor (FIGS. 8-10). The screws or fasteners can then be placed, for example under the guidance of a fluoroscope.

The muscle or tendon to be activated by the device is exposed and the tendon separated from its original muscle. The tether that is already affixed to the device is woven to the tendon in similar fashion to a typical tendon repair. In some embodiments, an incision of about the resting length of the device is made in the target area so as to attach the device to a bone on one end and to a bone/tendons/appendage on the other end. In other embodiments, the device is implanted through a small opening in the target area using a minimally invasive approach. For example, the tether or the elastic body may be routed under the skin by using a cannula until it reaches a bone, an appendage or a tendon.

The present invention is further illustrated by the following examples which should not be construed as limiting. The contents of all references, patents and published patent applications cited throughout this application, as well as the Figures and Tables, are incorporated herein by reference.

### Example 1

### Implantation of a Finger Extension Internal Splint Device

A patient presents with a complete brachial plexus injury of the left arm, resulting in the complete loss of the functions of wrist and finger extension in the left arm. The patient is determined to be a candidate for the implantation of a finger extension internal splint device.

An incision over the dorsum of the forearm is made to one side of the ultimate location of the device so that there will be no tension on the skin closure. An area on the radius is cleared off via subperiosteal dissection, providing adequate surface for placement of the base. Under fluoroscopic guidance, the screws are placed and the base is affixed to the bone.

Next, the tendons are followed as far distally as they can be harvested. A suture side to side tenodesis is performed, allowing for the anatomic cascade of the digits, but pulling them open evenly in a manner that would allow for effective encompassing of a particular object. The length of the tendon in reference to the radius is noted and the radius is marked at the position in full flexion and the position in full extension.

The device is then affixed so that full recoil will pull the fingers into appropriate extension. The tendons are pulled through the keyhole and woven back upon themselves with the ultimate positioning giving the effective hand open posture. The tension is then set, performed by the surgeon simply utilizing tactile feedback. Alternatively, the antagonist muscle can be stimulated to ensure that the recoil is strong enough to bring the fingers into full extension, but weak enough to be fully overcome in allowing the hand to make a fist with contraction of the healthy muscles. This tension is then locked in by appropriate adjustment of the “tension screw.” The skin is closed and the hand is immobilized in the open hand position (e.g., for 6 weeks or until the surgeon deems the tendon repairs “healed”). The patient is then encouraged to begin range of motion exercises and resume normal activity as tolerated.

The above description is for the purpose of teaching the person of ordinary skill in the art how to practice the present invention, and it is not intended to detail all those obvious modifications and variations of it which will become apparent to the skilled worker upon reading the description. It is intended, however, that all such obvious modifications and variations be included within the scope of the present invention, which is defined by the following claims. The claims are intended to cover the components and steps in any sequence which is effective to meet the objectives there intended, unless the context specifically indicates the contrary.

What is claimed is:

1. An internal dynamic splinting device, comprising:
an elastic body;
a first tissue-attaching component operatively connected to said elastic body, wherein said first tissue-attaching component is adapted to be attached to a bone; and
a second tissue-attaching component operatively connected to said elastic body, wherein said second tissue-attaching component is adapted to be attached to a bone or a soft tissue.

2. The internal dynamic splinting device of claim 1, wherein said elastic body comprises a first end, a second end and a resting length between said first end and said second end, wherein said elastic body has a resilience to extend to a stretched length when a stretching force is applied to said first end and/or said second end, and to return from said stretched length to said resting length after the removal of said stretching force, wherein said stretched length is at least 5% greater than said resting length.

3. The internal dynamic splinting device of claim 1, wherein said elastic body comprises a housing and an elastic component within said housing.

4. The internal dynamic splinting device of claim 3, wherein said elastic component comprises a spring.

5. The internal dynamic splinting device of claim 4, wherein said spring changes from a resting position to a stretched position when said elastic body extends from said resting length to said stretched length.

6. The internal dynamic splinting device of claim 4, wherein said spring changes from a resting position to a compressed position when said elastic body extends from said resting length to said stretched length.

7. The internal dynamic splinting device of claim 6, wherein said elastic body further comprises a tension adjustment component that controls tension within said elastic body.

8. The internal dynamic splinting device of claim 4, wherein said housing comprises an outer layer to prevent in-growth of tissues into said device.

9. The internal dynamic splinting device of claim 8, wherein said outer layer comprises silicone or polyurethane.

10. The internal dynamic splinting device of claim 2, wherein said first tissue-attaching component is connected to said first end of said elastic body and comprises a plate having at least one hole for mounting a fastening device that fastens said plate to a bone.

11. The internal dynamic splinting device of claim 2, wherein said second tissue-attaching component is connected to said second end of said elastic body and is adapted to attach to a soft tissue.

12. The internal dynamic splinting device of claim 1, wherein said elastic body comprises two or more elastic components and wherein each elastic component is engaged directly or indirectly with said first tissue-attachment component.

13. The internal dynamic splinting device of claim 12, wherein said two or more elastic components are springs.

14. A method for treating a condition involving muscle function deficiency, comprising:
making an incision at a target site on a subject requiring such treatment,
attaching said first tissue-attaching component of the internal dynamic splinting device of claim 1 to a first bone;
attaching said second tissue-attaching component of the internal dynamic splinting device of claim 1 to a second bone or a soft tissue; and
closing said incision.

15. The method of claim 14, wherein said condition is a condition of paralysis in which movement of one muscle group is unopposed and wherein said condition of paralysis is resulted from muscle injuries, peripheral nerve injuries, congenital conditions, lower spinal injuries, cervical spinal cord injuries, strokes or brain injuries.

16. The method of claim 14, wherein said condition is selected from the group consisting of foot drop, wrist drop and inability to extend fingers.

17. The method of claim 14, wherein said condition is a condition resulted from polio, multiple sclerosis, cerebral palsy, or paralysis related to West Nile virus, brain surgery, or Guillian Bane syndrome.

18. A method for compensating muscle function deficiency on a side of a joint, comprising:
attaching said first tissue-attaching component of the internal dynamic splinting device of claim 1 to a first bone on said side of said joint; and
attaching said second tissue-attaching component of the internal dynamic splinting device of claim 1 to a second bone or a soft tissue on said side of said joint,
wherein said internal dynamic splinting device is stretched from a resting length to a stretched length when an antagonist muscle on an opposite side of said joint contracts, and wherein said internal dynamic splinting device returns to said resting length when said antagonist muscle on said opposite side relaxes.

19. The method of claim 18, wherein said joint is a knee, ankle or wrist joint.

20. The method of claim 18, wherein said joint is an elbow, hip or shoulder joint.

21. An implantable splinting device, comprising:
an elastic body having a proximal end and a distal end, said elastic body comprises:
a housing;
a spring resided inside said housing; and
a rod connected to said spring, wherein said rod extends outside said housing from said distal end of said housing;
a first tissue-attaching component connected to said proximal end of said elastic body, wherein said first tissue-attaching component is adapted to be attached to a bone;
a tether connected to said rod; and
a second tissue-attaching component connected to said tether, wherein said second tissue-attaching component is adapted to be attached to a bone or a soft tissue.

22. The implantable splinting device of claim 21, wherein said device further comprises a tension adjustment component that controls tension of said spring.

23. The implantable splinting device of claim 22, wherein said tension adjustment component is located inside said housing.

24. The implantable splinting device of claim 22, wherein said tension adjustment component is located outside said housing.

25. The implantable splinting device of claim 21, wherein said tether is a non-elastic tether.

26. The implantable splinting device of claim 21, wherein said tether is an elastic tether.
